SEQUENCE LISTING (30 sequences)

SEQ ID NO: 1 - DNA, 999 bp, ARTIFICIAL
  LINC000518 ISOFORM 1
taagagtggggccctagtctgataggactggtgtccttacaagaagagggagagtcctcagagagtcctctctctcggcatggacacaaaagaaaagccatgtgaggacacagagagaaggtggctgtctacgagctaggaagaaaggcctcaccggaaaccaaccctcacagcagctccatcttggacttccagcctccggaactgtgagaaaataaatgtttgcaattcaggctcagagccactcagacattgggaagcaagtttgtcaagatgacagagaaccgaggtaatggattcgagtgatgaaacaggaagttcattcatgagtttttggccacacctccaaagtgacgacttagccagaaatgggataactgggtttccctacttctcttttatcatcctcaatgagagtgaccaaatattagagctagatggaaccttagtgaaaatctggctactcgtcccgtcccaccagcctgccacccatttcaagtttgaagagacaaagacacatggaccttatgtaattactggggattaccccaggagtctgtggcaaaagtcagcttcttccctccctgcttccccgccctgtctctggtactttctaccaacactgggctgtttctgtgatcacacttaagcgtacctaacctgcgaatgctgtatagaaggtgctaatgaacatgatttagctttaacactcagttttctaaagggacacgtgggggcagcaaatgtttaggcaaaaacaattccagttctagcctctactgtctacatatgtgtatacatttgggaaacgtttgggaaagggatatttgagagcttctttttcttttttgtggtttagttatttgatgatattgagattgtttctgagccatgtgcttcaacatcggattggggatttcagaaaaagttttagtcactgtgattccatttagcttccaaatgtgtctctgctaagagacttaaaagcactcataaata

SEQ ID NO: 2 - DNA, 906 bp, ARTIFICIAL
  LINC000518 ISOFORM 2
caagaagagggagagtcctcagagagtcctctctctcggcatggacacaaaagaaaagccatgtgaggacacagagagaaggtggctgtctacgagctaggaagaaaggcctcaccggaaaccaaccctcacagcagctccatcttggacttccagcctccggaactgtgagaaaataaatgtttgcaattcagggagctccacccagcagcagcgtttgacagagctgttcacttcctcttcctggagctgtggcttccagagcccatgctcagcagttcccctccttcttcgactgctcctctcttaggctcagagccactcagacattgggaagcaagtttgtcaagatgacagagaaccgaggtaatggattcgagtgatgaaacaggaagttcattcatgagtttttggccacacctccaaagtgacgacttagccagaaatgggataactgggtttccctacttctcttttatcatcctcaatgagagtgaccaaatattagagctagatggaaccttagtgaaaatctggctactcgtcccgtcccaccagcctgccacccatttcaagtttgaagagacaaagacacatggaccttatgtaattactggggattaccccaggagtctgtggcaaaagtcagcttcttccctccctgcttccccgccctgtctctggtactttctaccaacactgggctgtttctgtgatcacacttaagcgtacctaacctgcgaatgctgtatagaaggtgctaatgaacatgatttagctttaacactcagttttctaaagggacacgtgggggcagcaaatgtttaggcaaaaacaattccagttctagcctctactgtctacatatgtgtatacatttgggaaacgtttgggaaagggatattt

SEQ ID NO: 3 - DNA, 1548 bp, ARTIFICIAL
  LINC00518 ISOFORM 3
agtcctcagagagtcctctctctcggcatggacacaaaagaaaagccatgtgaggacacagagagaaggtggctgtctacgagctaggaagaaaggcctcaccggaaaccaaccctcacagcagctccatcttggacttccagcctccggaactgtgagaaaataaatgtttgcaattcaggtcggttgtattttgtgaaggccatcctagcaaatgaatactcctaacattgtctctttaagagctcaccagcctgaggtaggaatcattccatctgtgttactaatgagaccgctgaggatcaaaggggttttccaccacatccactcacctctacatggcgaaaaccaagggttcactctctgtcttcagggagctccacccagcagcagcgtttgacagagctgttcacttcctcttcctggagctgtggcttccagagcccatgctcagcagttcccctccttcttcgactgctcctctcttaggctcagagccactcagacattgggaagcaagtttgtcaagatgacagagaaccgaggtaatggattcgagtgatgaaacaggaagttcattcatgagtttttggccacacctccaaagtgacgacttagccagaaatgggataactgggtttccctacttctcttttatcatcctcaatgagagtgaccaaatattagagctagatggaaccttagtgaaaatctggctactcgtcccgtcccaccagcctgccacccatttcaagtttgaagagacaaagacacatggaccttatgtaattactggggattaccccaggagtctgtggcaaaagtcagcttcttccctccctgcttccccgccctgtctctggtactttctaccaacactgggctgtttctgtgatcacacttaagcgtacctaacctgcgaatgctgtatagaaggtgctaatgaacatgatttagctttaacactcagttttctaaagggacacgtgggggcagcaaatgtttaggcaaaaacaattccagttctagcctctactgtctacatatgtgtatacatttgggaaacgtttgggaaagggatatttgagagcttctttttcttttttgtggtttagttatttgatgatattgagattgtttctgagccatgtgcttcaacatcggattggggatttcagaaaaagttttagtcactgtgattccatttagcttccaaatgtgtctctgctaagagacttaaaagcactcataaatagcacgtgtgtcttctttgcagtgtttgctaattttgagtcacatctttttagaaaatcatgagatttggtgtcacagagactggaataaatatagtcaaacttattggtgaagatttcctttagctgttttcataatccatttccattgttatgattattgatgaataaaacattttctttaggtagatacttcttttttccccccaccttgatttaatgtttccactcttattgtcaagtttcttattactccctaataactctcaataaaataatgattcctgggagatta

SEQ ID NO: 4 - DNA, 1228 bp, ARTIFICIAL
  LINC00518 ISOFORM 5
tcagagagtcctctctctcggcatggacacaaaagaaaagccatgtgaggacacagagagaaggtggctgtctacgagctaggaagaaaggcctcaccggaaaccaaccctcacagcagctccatcttggacttccagcctccggaactgtgagaaaataaatgtttgcaattcaggtcggttgtattttgtgaaggccatcctagcaaatgaatactcctaacattgtctctttaagagctcaccagcctgaggtaggaatcattccatctgtgttactaatgagaccgctgaggatcaaaggggttttccaccacatccactcacctctacatggcgaaaaccaagggttcactctctgtcttcagggagctccacccagcagcagcgtttgacagagctgttcacttcctcttcctggagctgtggcttccagagcccatgctcagcagttcccctccttcttcgactgctcctctcttaggctcaaaacctgtaacaatgcatggcacacggaagatgtgagaatttgctgaataactgaatggcaccatgttgcctcacctaaggataatgactaagaataggaaaaatttactgtgcaaagatcagttcttaatagctgtctacatctcagaacaaaaaaaattagatgtaaaccattgcacaaaggattagatgaaatttttttgaaaaatcagacattggaggctcagagccactcagacattgggaagcaagtttgtcaagatgacagagaaccgaggtaatggattcgagtgatgaaacaggaagttcattcatgagtttttggccacacctccaaagtgacgacttagccagaaatgggataactgggtttccctacttctcttttatcatcctcaatgagagtgaccaaatattagagctagatggaaccttagtgaaaatctggctactcgtcccgtcccaccagcctgccacccatttcaagtttgaagagacaaagacacatggaccttatgtaattactggggattaccccaggagtctgtggcaaaagtcagcttcttccctccctgcttccccgccctgtctctggtactttctaccaacactgggctgtttctgtgatcacacttaagcgtacctaacctgcgaatgctgtatagaaggtgctaatgaacatgatttagctttaacactcagttttctaaagggacacgtgggggcagcaaatgtttag

SEQ ID NO: 5 - DNA, 853 bp, ARTIFICIAL
  LINC00518 ISOFORM 6
cagtgcctaggttttatgtagtgaatttggctaaaatggtagagctccctgattctaacctccctgcaggttttccaccacatccactcacctctacatggcgaaaaccaagggttcactctctgtcttcagggagctccacccagcagcagcgtttgacagagctgttcacttcctcttcctggagctgtggcttccagagcccatgctcagcagttcccctccttcttcgactgctcctctcttaggctcagagccactcagacattgggaagcaagtttgtcaagatgacagagaaccgaggtaatggattcgagtgatgaaacaggaagttcattcatgagtttttggccacacctccaaagtgacgacttagccagaaatgggataactgggtttccctacttctcttttatcatcctcaatgagagtgaccaaatattagagctagatggaaccttagtgaaaatctggctactcgtcccgtcccaccagcctgccacccatttcaagtttgaagagacaaagacacatggaccttatgtaattactggggattaccccaggagtctgtggcaaaagtcagcttcttccctccctgcttccccgccctgtctctggtactttctaccaacactgggctgtttctgtgatcacacttaagcgtacctaacctgcgaatgctgtatagaaggtgctaatgaacatgatttagctttaacactcagttttctaaagggacacgtgggggcagcaaatgtttaggcaaaaacaattccagttctagcctctactgtctacatatgtgtatacatttgggaaacgtttgggaaagggatatttgagagcttc

SEQ ID NO: 6 - DNA, 233 bp, ARTIFICIAL
  1ST EXON OF ISOFORM 1
taagagtggggccctagtctgataggactggtgtccttacaagaagagggagagtcctcagagagtcctctctctcggcatggacacaaaagaaaagccatgtgaggacacagagagaaggtggctgtctacgagctaggaagaaaggcctcaccggaaaccaaccctcacagcagctccatcttggacttccagcctccggaactgtgagaaaataaatgtttgcaattcag

SEQ ID NO: 7 - DNA, 4328 bp, ARTIFICIAL
  1ST INTRON OF ISOFORM 1
gtcggttgtattttgtgaaggccatcctagcaaatgaatactcctaacattgtctctttaagagctcaccagcctgaggtaggaatcattccatctgtgttactaatgagaccgctgaggatcaaaggggtgagtaaattgcttagcagtgcctaggttttatgtagtgaatttggctaaaatggtagagctccctgattctaacctccctgcaggttttccaccacatccactcacctctacatggcgaaaaccaagggttcactctctgtcttcagggagctccacccagcagcagcgtttgacagagctgttcacttcctcttcctggagctgtggcttccagagcccatgctcagcagttcccctccttcttcgactgctcctctcttaggtgagttttgttggtgtccttcatttccctgactctaaacattggagacctccacgtccagtcttgggcctcttccctgttcatccacaagcactccctaggtgacctcagccgtgcttatggttgttggccacagcgtccaaattgactttgctgcctccacacttgtccccctgccctgaccgagcagccaaggtgactctgttaagatacgaagcagatcacgccattgcttgactcaagcccctccaaagtttctgctctcagaggaaaagctaaggtcttttttttttttgagatggagtttcactcttgttgcccagactgcagtgcagtggtgtgatcttggctcactgcaacctccacttcccgggttcaagcgattctcttgcctcagcctcccaagtagctgggattacaggtgcacgccaccacgcccggctaatttttgtatttttagtagagatggggtttccccatgttggccaggctggtctcgaactcttgacctcaggtgatccacctgcctcggcttcccaatgtgctgggattacaggcatgagccacagtgcccagccaagctaaggtctttataatgtctcccttcggtaccaacagctccatcccccaactcccttcaattctcaatttctctttggcgtcctctgtagctcctctgtccttagctcactcatgcctaccacacaagccagccatgcttctgcctcagggcctttgcactcactggtttctctgcctggaatgtttctctggtaatgcagatgccaccatatccttggagacatccttatctcaactgcaaaccaccctccctctcagcactacctaacgtctttgtttatgtttttccttcagcccataccattcattatctaccatactatatattttacttaccttgtttattttctctcctctggaatacaaagtccacaagggcagagatttttgtcttttgttttcatggttctaggctcaaaacctgtaacaatgcatggcacacggaagatgtgagaatttgctgaataactgaatggcaccatgttgcctcacctaaggataatgactaagaataggaaaaatttactgtgcaaagatcagttcttaatagctgtctacatctcagaacaaaaaaaattagatgtaaaccattgcacaaaggattagatgaaatttttttgaaaaatcagacattggaggtaagtattgccataagccatataagcaaaacaatcaaatctctggagacactggaaaataagattgattgtaaatttgctgctgacgtttaccattgctctgcataaatgcaaaaggaagcgctggccaacttctcatgggtttccaagtccaggtgtctaaaatttggcagagctgggaggactgcttgaggccagtggtgcaaaataagcctgggcaacatagtgagattctgcctctaaaacaaaacaaaataaaagacaaaacaactaattctgttttttaaaaaaaaaaaacacacaacagcaaacttgactataaagattattgctgggcacggtggctcaggcctgtaatcccagcactttgggaggctgaggcgggtggatcacctgaggtcaggagctcaagaccagcctggccaacatggtgaaaccctgtctgtactaataatacaaaaaattagccgggcatggtggtgcatgcctgcaatcccagctactcgggaggatgaggcaggagaatcacttgaacctgggaggtagaggttgcagtgagccgagactgcgccactgcactccagcctgggcaacaagagcaaaactccgtgtccaaaaaaaaaaaaaaaaaaaaagattattatatataatcattcaaggcctgtatgactcagttcccttagaaaaatgtcataatttttatattactgaatattattggcgttatttgtgtagcccacttaagtgaagtcaataacatgattaagtggcatattatcttcatgtcagtcaaacgttatttggattttataagttagggtgagatacaaataagtgaaaatactttttctaatgaataatgatgaatctaaaataggattgacttggctgggcatagtggctcgtgcttgtaaccccaacactttgggaggctgaggcagtaggattacttgaaaccaggagtttgagaccagcctcggcaacaaagggagaactcttctctaataaaaataagaataaaaaattagccaggtgtggcaatgttcacctgtggtcccagctacttgggaagctgaggcaggaggatcgttggagcacaggagttcaagactgcagttagcggtgactgcactccagcctgggcaatagagcaagaccctgtctctaaaaaaaaaataataataaataggactggctcgcatatgtatgcaactattttgttaccagtgacagaatacatgtgatgatgttttctagtcttgtgactggtttcttagttgtatgttttatttttagaattgttgtttccaggctgtgttgtttggaaactcagctgacagatgtatgaatctgattccagagtctctgagagctggaaagggcctcagagatggttgggactgatactcttgcattacaggtcaggaaatacaacccagagacaagatgaaacttgcccaaggacataacagccagcctgtggctaggctgataccagattcctgttcttcccattccagtcgcatgctactcaggaaactcatcagcattcccgaatattgagtgtttcctacctgttacattagcagtttttgagaagggtatatattgtagttaaattaatcagctttgggaaaatatgggtgcagcatgggaatagagactgtcatttgagaatcattgcagaaatgacaaaaccctgagaatgaagaatgggcctgcaatagtcctggcactttgctcttccaccaaactgaactgtagctcaaaggacaatgacttcacgtctgtcactctaaagcctgttctaagatcacatcccctctggctctccctagatacttctgattatagttggtaatcatgcccattaaatgtctttggtttcttggtacacagagagaaggtaaataatgggttcatgatttaggtaagaatacacaaatatttttcacactataaatctgttttctattcagttaaatattagaccatctggtaatggagaaaatgtttgaaaatccacctagttaacctaaaagtttgcatttcctgcctttggggctttgaatttttaagttacatgtcttcgtagatggtgatcaggataaaactaatatctctcttagatgaatcaaaattcacccccttgggcaagggagggacccagcctttctttaaacatgtggttgcctgagagttgaacaaaactggaatagggagagaatggctttgccatgctgatcatggtattgtggagatttaagaactagtggccaggcgaggtggctcacgcctgtaatcccagcattgtgagaggccaaggcaggtggatcacttgaggccaggatttcgagaccagcccggccaacatgatgaccctgtttttactaaaaatacaaaaattagccgagtgtggtggtgtgtgcctgtagtcccaactgaggcatgagacttgcttcaacccaggaggcgggggttgcagtgagccgagattgtgccactgcactccagcctgggtgacagagtgagactctgtctcaaaaaaaaaaaaaaaaaaaactagtcagagctgttgaggtgttgaggcacctgctactgatacacactataaacgtggaagatgattttcatttttgtagtcatgagcaggatactgtataatgtataattgttggacattaaagaaaacaaactccttcttgtctccttag

SEQ ID NO: 8 - DNA, 766 bp, ARTIFICIAL
  2ND EXON OF ISOFORM 1
gctcagagccactcagacattgggaagcaagtttgtcaagatgacagagaaccgaggtaatggattcgagtgatgaaacaggaagttcattcatgagtttttggccacacctccaaagtgacgacttagccagaaatgggataactgggtttccctacttctcttttatcatcctcaatgagagtgaccaaatattagagctagatggaaccttagtgaaaatctggctactcgtcccgtcccaccagcctgccacccatttcaagtttgaagagacaaagacacatggaccttatgtaattactggggattaccccaggagtctgtggcaaaagtcagcttcttccctccctgcttccccgccctgtctctggtactttctaccaacactgggctgtttctgtgatcacacttaagcgtacctaacctgcgaatgctgtatagaaggtgctaatgaacatgatttagctttaacactcagttttctaaagggacacgtgggggcagcaaatgtttaggcaaaaacaattccagttctagcctctactgtctacatatgtgtatacatttgggaaacgtttgggaaagggatatttgagagcttctttttcttttttgtggtttagttatttgatgatattgagattgtttctgagccatgtgcttcaacatcggattggggatttcagaaaaagttttagtcactgtgattccatttagcttccaaatgtgtctctgctaagagacttaaaagcactcataaata

SEQ ID NO: 9 - DNA, 194 bp, ARTIFICIAL
  1ST EXON OF ISOFORM 2
caagaagagggagagtcctcagagagtcctctctctcggcatggacacaaaagaaaagccatgtgaggacacagagagaaggtggctgtctacgagctaggaagaaaggcctcaccggaaaccaaccctcacagcagctccatcttggacttccagcctccggaactgtgagaaaataaatgtttgcaattcag

SEQ ID NO: 10 - DNA, 278 bp, ARTIFICIAL
  1ST INTRON OF ISOFORM 2
gtcggttgtattttgtgaaggccatcctagcaaatgaatactcctaacattgtctctttaagagctcaccagcctgaggtaggaatcattccatctgtgttactaatgagaccgctgaggatcaaaggggtgagtaaattgcttagcagtgcctaggttttatgtagtgaatttggctaaaatggtagagctccctgattctaacctccctgcaggttttccaccacatccactcacctctacatggcgaaaaccaagggttcactctctgtcttcag

SEQ ID NO: 11 - DNA, 116 bp, ARTIFICIAL
  2ND EXON OF ISOFORM 2
ggagctccacccagcagcagcgtttgacagagctgttcacttcctcttcctggagctgtggcttccagagcccatgctcagcagttcccctccttcttcgactgctcctctcttag

SEQ ID NO: 12 - DNA, 3934 bp, ARTIFICIAL
  2ND INTRON OF ISOFORM 2
gtgagttttgttggtgtccttcatttccctgactctaaacattggagacctccacgtccagtcttgggcctcttccctgttcatccacaagcactccctaggtgacctcagccgtgcttatggttgttggccacagcgtccaaattgactttgctgcctccacacttgtccccctgccctgaccgagcagccaaggtgactctgttaagatacgaagcagatcacgccattgcttgactcaagcccctccaaagtttctgctctcagaggaaaagctaaggtcttttttttttttgagatggagtttcactcttgttgcccagactgcagtgcagtggtgtgatcttggctcactgcaacctccacttcccgggttcaagcgattctcttgcctcagcctcccaagtagctgggattacaggtgcacgccaccacgcccggctaatttttgtatttttagtagagatggggtttccccatgttggccaggctggtctcgaactcttgacctcaggtgatccacctgcctcggcttcccaatgtgctgggattacaggcatgagccacagtgcccagccaagctaaggtctttataatgtctcccttcggtaccaacagctccatcccccaactcccttcaattctcaatttctctttggcgtcctctgtagctcctctgtccttagctcactcatgcctaccacacaagccagccatgcttctgcctcagggcctttgcactcactggtttctctgcctggaatgtttctctggtaatgcagatgccaccatatccttggagacatccttatctcaactgcaaaccaccctccctctcagcactacctaacgtctttgtttatgtttttccttcagcccataccattcattatctaccatactatatattttacttaccttgtttattttctctcctctggaatacaaagtccacaagggcagagatttttgtcttttgttttcatggttctaggctcaaaacctgtaacaatgcatggcacacggaagatgtgagaatttgctgaataactgaatggcaccatgttgcctcacctaaggataatgactaagaataggaaaaatttactgtgcaaagatcagttcttaatagctgtctacatctcagaacaaaaaaaattagatgtaaaccattgcacaaaggattagatgaaatttttttgaaaaatcagacattggaggtaagtattgccataagccatataagcaaaacaatcaaatctctggagacactggaaaataagattgattgtaaatttgctgctgacgtttaccattgctctgcataaatgcaaaaggaagcgctggccaacttctcatgggtttccaagtccaggtgtctaaaatttggcagagctgggaggactgcttgaggccagtggtgcaaaataagcctgggcaacatagtgagattctgcctctaaaacaaaacaaaataaaagacaaaacaactaattctgttttttaaaaaaaaaaaacacacaacagcaaacttgactataaagattattgctgggcacggtggctcaggcctgtaatcccagcactttgggaggctgaggcgggtggatcacctgaggtcaggagctcaagaccagcctggccaacatggtgaaaccctgtctgtactaataatacaaaaaattagccgggcatggtggtgcatgcctgcaatcccagctactcgggaggatgaggcaggagaatcacttgaacctgggaggtagaggttgcagtgagccgagactgcgccactgcactccagcctgggcaacaagagcaaaactccgtgtccaaaaaaaaaaaaaaaaaaaaagattattatatataatcattcaaggcctgtatgactcagttcccttagaaaaatgtcataatttttatattactgaatattattggcgttatttgtgtagcccacttaagtgaagtcaataacatgattaagtggcatattatcttcatgtcagtcaaacgttatttggattttataagttagggtgagatacaaataagtgaaaatactttttctaatgaataatgatgaatctaaaataggattgacttggctgggcatagtggctcgtgcttgtaaccccaacactttgggaggctgaggcagtaggattacttgaaaccaggagtttgagaccagcctcggcaacaaagggagaactcttctctaataaaaataagaataaaaaattagccaggtgtggcaatgttcacctgtggtcccagctacttgggaagctgaggcaggaggatcgttggagcacaggagttcaagactgcagttagcggtgactgcactccagcctgggcaatagagcaagaccctgtctctaaaaaaaaaataataataaataggactggctcgcatatgtatgcaactattttgttaccagtgacagaatacatgtgatgatgttttctagtcttgtgactggtttcttagttgtatgttttatttttagaattgttgtttccaggctgtgttgtttggaaactcagctgacagatgtatgaatctgattccagagtctctgagagctggaaagggcctcagagatggttgggactgatactcttgcattacaggtcaggaaatacaacccagagacaagatgaaacttgcccaaggacataacagccagcctgtggctaggctgataccagattcctgttcttcccattccagtcgcatgctactcaggaaactcatcagcattcccgaatattgagtgtttcctacctgttacattagcagtttttgagaagggtatatattgtagttaaattaatcagctttgggaaaatatgggtgcagcatgggaatagagactgtcatttgagaatcattgcagaaatgacaaaaccctgagaatgaagaatgggcctgcaatagtcctggcactttgctcttccaccaaactgaactgtagctcaaaggacaatgacttcacgtctgtcactctaaagcctgttctaagatcacatcccctctggctctccctagatacttctgattatagttggtaatcatgcccattaaatgtctttggtttcttggtacacagagagaaggtaaataatgggttcatgatttaggtaagaatacacaaatatttttcacactataaatctgttttctattcagttaaatattagaccatctggtaatggagaaaatgtttgaaaatccacctagttaacctaaaagtttgcatttcctgcctttggggctttgaatttttaagttacatgtcttcgtagatggtgatcaggataaaactaatatctctcttagatgaatcaaaattcacccccttgggcaagggagggacccagcctttctttaaacatgtggttgcctgagagttgaacaaaactggaatagggagagaatggctttgccatgctgatcatggtattgtggagatttaagaactagtggccaggcgaggtggctcacgcctgtaatcccagcattgtgagaggccaaggcaggtggatcacttgaggccaggatttcgagaccagcccggccaacatgatgaccctgtttttactaaaaatacaaaaattagccgagtgtggtggtgtgtgcctgtagtcccaactgaggcatgagacttgcttcaacccaggaggcgggggttgcagtgagccgagattgtgccactgcactccagcctgggtgacagagtgagactctgtctcaaaaaaaaaaaaaaaaaaaactagtcagagctgttgaggtgttgaggcacctgctactgatacacactataaacgtggaagatgattttcatttttgtagtcatgagcaggatactgtataatgtataattgttggacattaaagaaaacaaactccttcttgtctccttag

SEQ ID NO: 13 - DNA, 596 bp, ARTIFICIAL
  3RD EXON OF ISOFORM 2
gctcagagccactcagacattgggaagcaagtttgtcaagatgacagagaaccgaggtaatggattcgagtgatgaaacaggaagttcattcatgagtttttggccacacctccaaagtgacgacttagccagaaatgggataactgggtttccctacttctcttttatcatcctcaatgagagtgaccaaatattagagctagatggaaccttagtgaaaatctggctactcgtcccgtcccaccagcctgccacccatttcaagtttgaagagacaaagacacatggaccttatgtaattactggggattaccccaggagtctgtggcaaaagtcagcttcttccctccctgcttccccgccctgtctctggtactttctaccaacactgggctgtttctgtgatcacacttaagcgtacctaacctgcgaatgctgtatagaaggtgctaatgaacatgatttagctttaacactcagttttctaaagggacacgtgggggcagcaaatgtttaggcaaaaacaattccagttctagcctctactgtctacatatgtgtatacatttgggaaacgtttgggaaagggatattt

SEQ ID NO: 14 - DNA, 310 bp, ARTIFICIAL
  1ST EXON OF ISOFORM 3
agtcctcagagagtcctctctctcggcatggacacaaaagaaaagccatgtgaggacacagagagaaggtggctgtctacgagctaggaagaaaggcctcaccggaaaccaaccctcacagcagctccatcttggacttccagcctccggaactgtgagaaaataaatgtttgcaattcaggtcggttgtattttgtgaaggccatcctagcaaatgaatactcctaacattgtctctttaagagctcaccagcctgaggtaggaatcattccatctgtgttactaatgagaccgctgaggatcaaaggg

SEQ ID NO: 15 - DNA, 86 bp, ARTIFICIAL
  1ST INTRON OF ISOFORM 3
gtgagtaaattgcttagcagtgcctaggttttatgtagtgaatttggctaaaatggtagagctccctgattctaacctccctgcag

SEQ ID NO: 16 - DNA, 179 bp, ARTIFICIAL
  2ND EXON OF ISOFORM 3
gttttccaccacatccactcacctctacatggcgaaaaccaagggttcactctctgtcttcagggagctccacccagcagcagcgtttgacagagctgttcacttcctcttcctggagctgtggcttccagagcccatgctcagcagttcccctccttcttcgactgctcctctcttag

SEQ ID NO: 17 - DNA, 3934 bp, ARTIFICIAL
  2ND INTRON OF ISOFORM 3
ctaaggagacaagaaggagtttgttttctttaatgtccaacaattatacattatacagtatcctgctcatgactacaaaaatgaaaatcatcttccacgtttatagtgtgtatcagtagcaggtgcctcaacacctcaacagctctgactagttttttttttttttttttttgagacagagtctcactctgtcacccaggctggagtgcagtggcacaatctcggctcactgcaacccccgcctcctgggttgaagcaagtctcatgcctcagttgggactacaggcacacaccaccacactcggctaatttttgtatttttagtaaaaacagggtcatcatgttggccgggctggtctcgaaatcctggcctcaagtgatccacctgccttggcctctcacaatgctgggattacaggcgtgagccacctcgcctggccactagttcttaaatctccacaataccatgatcagcatggcaaagccattctctccctattccagttttgttcaactctcaggcaaccacatgtttaaagaaaggctgggtccctcccttgcccaagggggtgaattttgattcatctaagagagatattagttttatcctgatcaccatctacgaagacatgtaacttaaaaattcaaagccccaaaggcaggaaatgcaaacttttaggttaactaggtggattttcaaacattttctccattaccagatggtctaatatttaactgaatagaaaacagatttatagtgtgaaaaatatttgtgtattcttacctaaatcatgaacccattatttaccttctctctgtgtaccaagaaaccaaagacatttaatgggcatgattaccaactataatcagaagtatctagggagagccagaggggatgtgatcttagaacaggctttagagtgacagacgtgaagtcattgtcctttgagctacagttcagtttggtggaagagcaaagtgccaggactattgcaggcccattcttcattctcagggttttgtcatttctgcaatgattctcaaatgacagtctctattcccatgctgcacccatattttcccaaagctgattaatttaactacaatatatacccttctcaaaaactgctaatgtaacaggtaggaaacactcaatattcgggaatgctgatgagtttcctgagtagcatgcgactggaatgggaagaacaggaatctggtatcagcctagccacaggctggctgttatgtccttgggcaagtttcatcttgtctctgggttgtatttcctgacctgtaatgcaagagtatcagtcccaaccatctctgaggccctttccagctctcagagactctggaatcagattcatacatctgtcagctgagtttccaaacaacacagcctggaaacaacaattctaaaaataaaacatacaactaagaaaccagtcacaagactagaaaacatcatcacatgtattctgtcactggtaacaaaatagttgcatacatatgcgagccagtcctatttattattattttttttttagagacagggtcttgctctattgcccaggctggagtgcagtcaccgctaactgcagtcttgaactcctgtgctccaacgatcctcctgcctcagcttcccaagtagctgggaccacaggtgaacattgccacacctggctaattttttattcttatttttattagagaagagttctccctttgttgccgaggctggtctcaaactcctggtttcaagtaatcctactgcctcagcctcccaaagtgttggggttacaagcacgagccactatgcccagccaagtcaatcctattttagattcatcattattcattagaaaaagtattttcacttatttgtatctcaccctaacttataaaatccaaataacgtttgactgacatgaagataatatgccacttaatcatgttattgacttcacttaagtgggctacacaaataacgccaataatattcagtaatataaaaattatgacatttttctaagggaactgagtcatacaggccttgaatgattatatataataatctttttttttttttttttttttggacacggagttttgctcttgttgcccaggctggagtgcagtggcgcagtctcggctcactgcaacctctacctcccaggttcaagtgattctcctgcctcatcctcccgagtagctgggattgcaggcatgcaccaccatgcccggctaattttttgtattattagtacagacagggtttcaccatgttggccaggctggtcttgagctcctgacctcaggtgatccacccgcctcagcctcccaaagtgctgggattacaggcctgagccaccgtgcccagcaataatctttatagtcaagtttgctgttgtgtgttttttttttttaaaaaacagaattagttgttttgtcttttattttgttttgttttagaggcagaatctcactatgttgcccaggcttattttgcaccactggcctcaagcagtcctcccagctctgccaaattttagacacctggacttggaaacccatgagaagttggccagcgcttccttttgcatttatgcagagcaatggtaaacgtcagcagcaaatttacaatcaatcttattttccagtgtctccagagatttgattgttttgcttatatggcttatggcaatacttacctccaatgtctgatttttcaaaaaaatttcatctaatcctttgtgcaatggtttacatctaattttttttgttctgagatgtagacagctattaagaactgatctttgcacagtaaatttttcctattcttagtcattatccttaggtgaggcaacatggtgccattcagttattcagcaaattctcacatcttccgtgtgccatgcattgttacaggttttgagcctagaaccatgaaaacaaaagacaaaaatctctgcccttgtggactttgtattccagaggagagaaaataaacaaggtaagtaaaatatatagtatggtagataatgaatggtatgggctgaaggaaaaacataaacaaagacgttaggtagtgctgagagggagggtggtttgcagttgagataaggatgtctccaaggatatggtggcatctgcattaccagagaaacattccaggcagagaaaccagtgagtgcaaaggccctgaggcagaagcatggctggcttgtgtggtaggcatgagtgagctaaggacagaggagctacagaggacgccaaagagaaattgagaattgaagggagttgggggatggagctgttggtaccgaagggagacattataaagaccttagcttggctgggcactgtggctcatgcctgtaatcccagcacattgggaagccgaggcaggtggatcacctgaggtcaagagttcgagaccagcctggccaacatggggaaaccccatctctactaaaaatacaaaaattagccgggcgtggtggcgtgcacctgtaatcccagctacttgggaggctgaggcaagagaatcgcttgaacccgggaagtggaggttgcagtgagccaagatcacaccactgcactgcagtctgggcaacaagagtgaaactccatctcaaaaaaaaaaaagaccttagcttttcctctgagagcagaaactttggaggggcttgagtcaagcaatggcgtgatctgcttcgtatcttaacagagtcaccttggctgctcggtcagggcagggggacaagtgtggaggcagcaaagtcaatttggacgctgtggccaacaaccataagcacggctgaggtcacctagggagtgcttgtggatgaacagggaagaggcccaagactggacgtggaggtctccaatgtttagagtcagggaaatgaaggacaccaacaaaactcac

SEQ ID NO: 18 - DNA, 1059 bp, ARTIFICIAL
  3RD EXON OF ISOFORM 3
gctcagagccactcagacattgggaagcaagtttgtcaagatgacagagaaccgaggtaatggattcgagtgatgaaacaggaagttcattcatgagtttttggccacacctccaaagtgacgacttagccagaaatgggataactgggtttccctacttctcttttatcatcctcaatgagagtgaccaaatattagagctagatggaaccttagtgaaaatctggctactcgtcccgtcccaccagcctgccacccatttcaagtttgaagagacaaagacacatggaccttatgtaattactggggattaccccaggagtctgtggcaaaagtcagcttcttccctccctgcttccccgccctgtctctggtactttctaccaacactgggctgtttctgtgatcacacttaagcgtacctaacctgcgaatgctgtatagaaggtgctaatgaacatgatttagctttaacactcagttttctaaagggacacgtgggggcagcaaatgtttaggcaaaaacaattccagttctagcctctactgtctacatatgtgtatacatttgggaaacgtttgggaaagggatatttgagagcttctttttcttttttgtggtttagttatttgatgatattgagattgtttctgagccatgtgcttcaacatcggattggggatttcagaaaaagttttagtcactgtgattccatttagcttccaaatgtgtctctgctaagagacttaaaagcactcataaatagcacgtgtgtcttctttgcagtgtttgctaattttgagtcacatctttttagaaaatcatgagatttggtgtcacagagactggaataaatatagtcaaacttattggtgaagatttcctttagctgttttcataatccatttccattgttatgattattgatgaataaaacattttctttaggtagatacttcttttttccccccaccttgatttaatgtttccactcttattgtcaagtttcttattactccctaataactctcaataaaataatgattcctgggagatta

SEQ ID NO: 19 - DNA, 305 bp, ARTIFICIAL
  1ST EXON OF ISOFORM 5
tcagagagtcctctctctcggcatggacacaaaagaaaagccatgtgaggacacagagagaaggtggctgtctacgagctaggaagaaaggcctcaccggaaaccaaccctcacagcagctccatcttggacttccagcctccggaactgtgagaaaataaatgtttgcaattcaggtcggttgtattttgtgaaggccatcctagcaaatgaatactcctaacattgtctctttaagagctcaccagcctgaggtaggaatcattccatctgtgttactaatgagaccgctgaggatcaaaggg

SEQ ID NO: 20 - DNA, 86 bp, ARTIFICIAL; identical to SEQ ID NO: 15
  1ST INTRON OF ISOFORM 5

SEQ ID NO: 21 - DNA, 179 bp, ARTIFICIAL; identical to SEQ ID NO: 16
  2ND EXON OF ISOFORM 5

SEQ ID NO: 22 - DNA, 994 bp, ARTIFICIAL
  2ND INTRON OF ISOFORM 5
gtgagttttgttggtgtccttcatttccctgactctaaacattggagacctccacgtccagtcttgggcctcttccctgttcatccacaagcactccctaggtgacctcagccgtgcttatggttgttggccacagcgtccaaattgactttgctgcctccacacttgtccccctgccctgaccgagcagccaaggtgactctgttaagatacgaagcagatcacgccattgcttgactcaagcccctccaaagtttctgctctcagaggaaaagctaaggtcttttttttttttgagatggagtttcactcttgttgcccagactgcagtgcagtggtgtgatcttggctcactgcaacctccacttcccgggttcaagcgattctcttgcctcagcctcccaagtagctgggattacaggtgcacgccaccacgcccggctaatttttgtatttttagtagagatggggtttccccatgttggccaggctggtctcgaactcttgacctcaggtgatccacctgcctcggcttcccaatgtgctgggattacaggcatgagccacagtgcccagccaagctaaggtctttataatgtctcccttcggtaccaacagctccatcccccaactcccttcaattctcaatttctctttggcgtcctctgtagctcctctgtccttagctcactcatgcctaccacacaagccagccatgcttctgcctcagggcctttgcactcactggtttctctgcctggaatgtttctctggtaatgcagatgccaccatatccttggagacatccttatctcaactgcaaaccaccctccctctcagcactacctaacgtctttgtttatgtttttccttcagcccataccattcattatctaccatactatatattttacttaccttgtttattttctctcctctggaatacaaagtccacaagggcagagatttttgtcttttgttttcatggttctag

SEQ ID NO: 23 - DNA, 226 bp, ARTIFICIAL
  3RD EXON OF ISOFORM 5
gctcaaaacctgtaacaatgcatggcacacggaagatgtgagaatttgctgaataactgaatggcaccatgttgcctcacctaaggataatgactaagaataggaaaaatttactgtgcaaagatcagttcttaatagctgtctacatctcagaacaaaaaaaattagatgtaaaccattgcacaaaggattagatgaaatttttttgaaaaatcagacattggag

SEQ ID NO: 24 - DNA, 2714 bp, ARTIFICIAL
  3RD INTRON OF ISOFORM 5
gtaagtattgccataagccatataagcaaaacaatcaaatctctggagacactggaaaataagattgattgtaaatttgctgctgacgtttaccattgctctgcataaatgcaaaaggaagcgctggccaacttctcatgggtttccaagtccaggtgtctaaaatttggcagagctgggaggactgcttgaggccagtggtgcaaaataagcctgggcaacatagtgagattctgcctctaaaacaaaacaaaataaaagacaaaacaactaattctgttttttaaaaaaaaaaaacacacaacagcaaacttgactataaagattattgctgggcacggtggctcaggcctgtaatcccagcactttgggaggctgaggcgggtggatcacctgaggtcaggagctcaagaccagcctggccaacatggtgaaaccctgtctgtactaataatacaaaaaattagccgggcatggtggtgcatgcctgcaatcccagctactcgggaggatgaggcaggagaatcacttgaacctgggaggtagaggttgcagtgagccgagactgcgccactgcactccagcctgggcaacaagagcaaaactccgtgtccaaaaaaaaaaaaaaaaaaaaagattattatatataatcattcaaggcctgtatgactcagttcccttagaaaaatgtcataatttttatattactgaatattattggcgttatttgtgtagcccacttaagtgaagtcaataacatgattaagtggcatattatcttcatgtcagtcaaacgttatttggattttataagttagggtgagatacaaataagtgaaaatactttttctaatgaataatgatgaatctaaaataggattgacttggctgggcatagtggctcgtgcttgtaaccccaacactttgggaggctgaggcagtaggattacttgaaaccaggagtttgagaccagcctcggcaacaaagggagaactcttctctaataaaaataagaataaaaaattagccaggtgtggcaatgttcacctgtggtcccagctacttgggaagctgaggcaggaggatcgttggagcacaggagttcaagactgcagttagcggtgactgcactccagcctgggcaatagagcaagaccctgtctctaaaaaaaaaataataataaataggactggctcgcatatgtatgcaactattttgttaccagtgacagaatacatgtgatgatgttttctagtcttgtgactggtttcttagttgtatgttttatttttagaattgttgtttccaggctgtgttgtttggaaactcagctgacagatgtatgaatctgattccagagtctctgagagctggaaagggcctcagagatggttgggactgatactcttgcattacaggtcaggaaatacaacccagagacaagatgaaacttgcccaaggacataacagccagcctgtggctaggctgataccagattcctgttcttcccattccagtcgcatgctactcaggaaactcatcagcattcccgaatattgagtgtttcctacctgttacattagcagtttttgagaagggtatatattgtagttaaattaatcagctttgggaaaatatgggtgcagcatgggaatagagactgtcatttgagaatcattgcagaaatgacaaaaccctgagaatgaagaatgggcctgcaatagtcctggcactttgctcttccaccaaactgaactgtagctcaaaggacaatgacttcacgtctgtcactctaaagcctgttctaagatcacatcccctctggctctccctagatacttctgattatagttggtaatcatgcccattaaatgtctttggtttcttggtacacagagagaaggtaaataatgggttcatgatttaggtaagaatacacaaatatttttcacactataaatctgttttctattcagttaaatattagaccatctggtaatggagaaaatgtttgaaaatccacctagttaacctaaaagtttgcatttcctgcctttggggctttgaatttttaagttacatgtcttcgtagatggtgatcaggataaaactaatatctctcttagatgaatcaaaattcacccccttgggcaagggagggacccagcctttctttaaacatgtggttgcctgagagttgaacaaaactggaatagggagagaatggctttgccatgctgatcatggtattgtggagatttaagaactagtggccaggcgaggtggctcacgcctgtaatcccagcattgtgagaggccaaggcaggtggatcacttgaggccaggatttcgagaccagcccggccaacatgatgaccctgtttttactaaaaatacaaaaattagccgagtgtggtggtgtgtgcctgtagtcccaactgaggcatgagacttgcttcaacccaggaggcgggggttgcagtgagccgagattgtgccactgcactccagcctgggtgacagagtgagactctgtctcaaaaaaaaaaaaaaaaaaaactagtcagagctgttgaggtgttgaggcacctgctactgatacacactataaacgtggaagatgattttcatttttgtagtcatgagcaggatactgtataatgtataattgttggacattaaagaaaacaaactccttcttgtctccttag

SEQ ID NO: 25 - DNA, 518 bp, ARTIFICIAL
  4TH EXON OF ISOFORM 5
gctcagagccactcagacattgggaagcaagtttgtcaagatgacagagaaccgaggtaatggattcgagtgatgaaacaggaagttcattcatgagtttttggccacacctccaaagtgacgacttagccagaaatgggataactgggtttccctacttctcttttatcatcctcaatgagagtgaccaaatattagagctagatggaaccttagtgaaaatctggctactcgtcccgtcccaccagcctgccacccatttcaagtttgaagagacaaagacacatggaccttatgtaattactggggattaccccaggagtctgtggcaaaagtcagcttcttccctccctgcttccccgccctgtctctggtactttctaccaacactgggctgtttctgtgatcacacttaagcgtacctaacctgcgaatgctgtatagaaggtgctaatgaacatgatttagctttaacactcagttttctaaagggacacgtgggggcagcaaatgtttag

SEQ ID NO: 26 - DNA, 248 bp, ARTIFICIAL
  1ST EXON OF ISOFORM 6
cagtgcctaggttttatgtagtgaatttggctaaaatggtagagctccctgattctaacctccctgcaggttttccaccacatccactcacctctacatggcgaaaaccaagggttcactctctgtcttcagggagctccacccagcagcagcgtttgacagagctgttcacttcctcttcctggagctgtggcttccagagcccatgctcagcagttcccctccttcttcgactgctcctctcttag

SEQ ID NO: 27 - DNA, 3934 bp, ARTIFICIAL; identical to SEQ ID NO: 12
  1ST INTRON OF ISOFORM 6

SEQ ID NO: 28 - DNA, 605 bp, ARTIFICIAL
  2ND EXON OF ISOFORM 6
gctcagagccactcagacattgggaagcaagtttgtcaagatgacagagaaccgaggtaatggattcgagtgatgaaacaggaagttcattcatgagtttttggccacacctccaaagtgacgacttagccagaaatgggataactgggtttccctacttctcttttatcatcctcaatgagagtgaccaaatattagagctagatggaaccttagtgaaaatctggctactcgtcccgtcccaccagcctgccacccatttcaagtttgaagagacaaagacacatggaccttatgtaattactggggattaccccaggagtctgtggcaaaagtcagcttcttccctccctgcttccccgccctgtctctggtactttctaccaacactgggctgtttctgtgatcacacttaagcgtacctaacctgcgaatgctgtatagaaggtgctaatgaacatgatttagctttaacactcagttttctaaagggacacgtgggggcagcaaatgtttaggcaaaaacaattccagttctagcctctactgtctacatatgtgtatacatttgggaaacgtttgggaaagggatatttgagagcttc

SEQ ID NO: 29 - DNA, 16 bp, ARTIFICIAL
  GAPMER 1
ccgacctgaattgcaa

SEQ ID NO: 30 - DNA, 16 bp, ARTIFICIAL
  GAPMER 2
gtagaggctagaactg

(19) United States
(12) Patent Application Publication
(10) Pub. No.: US 2023/0090446 A1
(43) Pub. Date: Mar. 23, 2023

(54) ANTISENSE OLIGONUCLEOTIDE TARGETING LINC00518 FOR TREATING MELANOMA

(71) Applicants: UNIVERSITE DE STRASBOURG, STRASBOURG (FR); CENTRE NATIONAL DE LA RECHERCHE SCIENTIFIQUE, PARIS (FR); INSERM (INSTITUT NATIONAL DE LA SANTE ET DE LA RECHERCH EMEDICALE), PARIS (FR)

(72) Inventors: IRWIN DAVIDSON, STRASBOURG (FR); GIOVANNI GAMBI, STRASBOURG (FR)

(21) Appl. No.: 17/795,560

(22) PCT Filed: Jan. 28, 2021
(86) PCT No.: PCT/EP2021/051971

§ 371 Date: Jul. 27, 2022

(30) Foreign Application Priority Data
Jan. 28, 2020 (EP) 20305074.5

(51) Int. Cl. C12N 15/113; A61K 31/437; A61K 31/713; A61P 35/00; A61P 17/00

(52) U.S. Cl. CPC C12N 15/1135; A61K 31/437; A61K 31/713; A61P 35/00; A61P 17/00; C12N 2310/11; C12N 2310/341; C12N 2310/3231; C12N 2320/31

(57) ABSTRACT

The present invention relates to the use of an antisense oligonucleotide targeting LINC00518 for the treatment of melanoma.

### FIELD OF THE INVENTION

The present invention relates to the field of oncology, in particular to the treatment of melanoma.

### BACKGROUND OF THE INVENTION

Malignant melanoma is a highly aggressive cancer that develops from pigment-producing cells known as melanocytes.

At early stages, treatment is typically removal by surgery. Under conditions where no dissemination of the primary lesion has occurred, most people are cured by this surgery. For those in whom melanoma has spread, immune-checkpoint therapy, targeted inhibitor therapy, or more rarely chemotherapy may improve survival. With treatment, the five-year survival rates in the United States is 99% among those with localized disease, 65% when spread has occurred to lymph nodes, and 25% among those with distant spread.

Typical treatments can be surgery, treatment with high dose of interferon with severe side effects, treatment by chemotherapy with dacarbazine, targeted therapy for BRAF mutated melanoma with BRAF inhibitors such as vemurafenib and dabrafenib, MEK inhibitors such as trametinib, cobimetinib and binimetinib, and C-Kit inhibitors. Treatment of BRAF mutated melanoma with BRAF and MEK inhibitors leads to an early beneficial effect, but melanoma tumors most often develop resistance to these inhibitors such that the therapy is no longer effective. In addition, immunotherapy including treatments with cytokines, immune checkpoint inhibitors, and adoptive T-cell transfer are also employed. More particularly, immune check point inhibitors include anti-CTLA-4 monoclonal antibodies (ipilimumab and tremelimumab), anti-PD-1 (pembrolizumab, pidilizumab, and nivolumab) and PD-L1 antibodies are also used as first line treatment of stage IV metastatic disease. However, although patients can show long term remission making immune-checkpoint therapy the best available option for advanced melanoma, at best only 40% of patients are responders.

The development of targeted therapies and immunotherapies led to a substantial improvement in overall survival of patients. However, the long-term efficacy of such treatments is limited by side effects, rapidly emerging resistance to treatment in the case of kinase inhibitors and the low fraction of patients responding to immunotherapy.

Therefore, there is still a need for new therapeutic options for treating melanoma that act independently of current therapies by different mechanisms or that can be used in combination with current therapies to increase their efficiency and/or the pool of concerned patients.

### SUMMARY OF THE INVENTION

The present invention provides a new strategy for the treatment of melanoma. Indeed, the inventors identified that antisense oligonucleotides targeting LINC00518 both decrease melanoma cancer cell proliferation and increase melanoma cancer cell apoptosis, both independently of and in cooperation with BRAF inhibitor such as Vemurafenib. Surprisingly, LINC00518 inhibition synergizes with MAPK pathway targeting to induce apoptosis, especially when the antisense oligonucleotides targeting LINC00518 is combined with a B-Raf inhibitor such as Vemurafenib or Dabrafenib and/or a MEK inhibitor such as Trametinib. In addition, antisense oligonucleotides targeting LINC00518 could be used for preventing, decreasing or delaying the appearance of resistance to a targeted therapy and may allow use of the targeted therapies at lower doses, thereby decreasing their side effects. Finally, the inventors demonstrate that LINC00518 localises to mitochondria and regulates oxidative phosphorylation in melanoma cells. Therefore, there is a great interest to use a molecule able to inhibit LINC00518 in mitochondria and antisense oligonucleotides are perfectly adapted to this goal.

Therefore, the present invention relates to an antisense oligonucleotide inhibiting expression of LINC00518 or a pharmaceutical composition comprising such an antisense oligonucleotide for use in the treatment of melanoma; the use of an antisense oligonucleotide inhibiting expression of LINC00518 or a pharmaceutical composition comprising such an antisense oligonucleotide for the manufacture of a medicament for use in the treatment of melanoma; and a method for treating a subject having a melanoma comprising administering a therapeutically effective amount of an antisense oligonucleotide inhibiting expression of LINC00518 or a pharmaceutical composition comprising such an antisense oligonucleotide.

In a preferred aspect, the antisense oligonucleotide increases cancer cell apoptosis. In another preferred aspect, the antisense oligonucleotide decreases cancer cell proliferation. In a more preferred aspect, the antisense oligonucleotide increases cancer cell apoptosis and decreases cancer cell proliferation.

In one aspect, the expression of LINC00518 is inhibited in the nucleus, the cytoplasm or/and he mitochondria. In another aspect, the expression of LINC00518 is inhibited in the nucleus, and optionally in the cytoplasm. In a further aspect, the expression of LINC00518 is inhibited in the mitochondria.

Optionally, the melanoma is a resistant melanoma, in particular a melanoma resistant to targeted therapy, chemotherapy or immune checkpoint therapy. Optionally, the melanoma is an advanced melanoma or a metastatic melanoma.

Optionally, the antisense oligonucleotide inhibiting expression of LINC00518 or the pharmaceutical composition comprising it is used in combination with a therapeutic agent used for the treatment of melanoma. Optionally, the pharmaceutical composition comprises the antisense oligonucleotide inhibiting expression of LINC00518 and the therapeutic agent. Optionally, the present invention relates to a method for treating a subject having a melanoma comprising administering a therapeutically effective amount of an antisense oligonucleotide inhibiting expression of LINC00518 or a pharmaceutical composition comprising such an antisense oligonucleotide, and administering a therapeutically effective amount of a therapeutic agent.

Optionally, the therapeutic agent used for the treatment of melanoma can be selected from the group consisting of a BRAF inhibitor, a C-Kit inhibitor, a chemotherapy, a MEK inhibitor and immunotherapy, preferably a BRAF inhibitor. More specifically, the therapeutic agent used for the treatment of melanoma can be selected from the group consisting of dabrafenib, vemurafenib, encorafenib, trametinib, binimetinib, temozolomide, dacarbazine, an anti-PD-1 antibody such as pembrolizumab, pidilizumab, and nivolumab, an anti-CTLA-4 such as ipilimumab and tremelimumab, a TKR agonist, a CD40 agonist and an anti-PD-L1 antibody, preferably dabrafenib, vemurafenib and encorafenib.

Optionally, the therapeutic agent used for the treatment of melanoma is selected from the group consisting of a BRAF inhibitor, a C-Kit inhibitor, and a MEK inhibitor, preferably a BRAF inhibitor. More specifically, the therapeutic agent used for the treatment of melanoma can be selected from the group consisting of dabrafenib, vemurafenib, encorafenib, trametinib, and binimetinib, preferably dabrafenib, vemurafenib and encorafenib. Optionally, the therapeutic agent used for the treatment of melanoma is to be administered at a sub-therapeutic amount.

Optionally, the therapeutic agent used for the treatment of melanoma is selected from the group consisting of a chemotherapy and immunotherapy. More specifically, the therapeutic agent used for the treatment of melanoma can be selected from the group consisting of temozolomide, dacarbazine, an anti-PD-1 antibody such as pembrolizumab, pidilizumab, and nivolumab, an anti-CTLA-4 such as ipilimumab and tremelimumab, a TKR agonist, a CD40 agonist and an anti-PD-L1 antibody.

In one specific aspect, the antisense oligonucleotide induces a RNase H mediated degradation. For instance, the antisense oligonucleotide can be a Gapmer, in particular a LNA gapmer, a MOE gapmer, a mixed wing Gapmer or an alternating flank gapmer.

In one aspect, the antisense oligonucleotide comprises a contiguous nucleotide sequence of 10 to 30 nucleotides in length wherein the contiguous nucleotide sequence is at least 90, 95, 96, 97, 98, 99 or 100 percent complementary to exon 4 of LINC00518.

In another aspect, the antisense oligonucleotide comprises a contiguous nucleotide sequence of 10 to 30 nucleotides in length wherein the contiguous nucleotide sequence is at least 90, 95, 96, 97, 98, 99 or 100 percent complementary to exon 1 of LINC00518.

In another aspect, the antisense oligonucleotide comprises a contiguous nucleotide sequence of 10 to 30 nucleotides in length wherein the contiguous nucleotide sequence is at least 90, 95, 96, 97, 98, 99 or 100 percent complementary to exon 2 of LINC00518.

In another aspect, the antisense oligonucleotide comprises a contiguous nucleotide sequence of 10 to 30 nucleotides in length wherein the contiguous nucleotide sequence is at least 90, 95, 96, 97, 98, 99 or 100 percent complementary to exon 3 of LINC00518.

In another aspect, the present invention relates to an antisense oligonucleotide as defined herein for use for preventing, decreasing or delaying the appearance of a resistance to a targeted therapy. It also relates to the use of an antisense oligonucleotide as defined herein for the manufacture of a medicament for preventing, decreasing or delaying the appearance of a resistance to a targeted therapy. It further relates to a method for preventing, decreasing or delaying the appearance of a resistance to a targeted therapy in a subject, comprising administering a therapeutically effective amount of an antisense oligonucleotide as defined herein to the subject and a therapeutically or sub-therapeutically effective amount of the targeted therapy, thereby preventing, decreasing or delaying the appearance of a resistance to the targeted therapy in the subject. Optionally, the targeted therapy is selected from the group consisting of a BRAF inhibitor, a C-Kit inhibitor, and a MEK inhibitor, preferably a BRAF inhibitor. More specifically, the targeted therapy can be selected from the group consisting of dabrafenib, vemurafenib, encorafenib, trametinib, and binimetinib, preferably dabrafenib, vemurafenib and encorafenib.

### BRIEF DESCRIPTION OF THE FIGURES

FIG. 1: LINC00518 expression in normal tissues and human tumors

(A-B) The Genotype-Tissue Expression (GTEx) and The Cancer Genome Atlas (TCGA) RNA sequencing data were analyzed for LINC00518 expression across normal tissue and tumor samples respectively. (C) LINC00518 expression levels in normal skin from GTEx and Skin Cutaneous Melanoma (SKCM) from TCGA were compared. Significant difference was evaluated by one-way Anova.

FIG. 2: LINC00518 expression in melanoma correlates with patient outcome

SKCM RNAseq expression data for LINC00518 were extracted from the TCGA database and correlated with patient overall survival (A) and Clark score of lesions (B). In A, significant difference between patients expressing high and low levels of LINC00518 was evaluated by Log-rank test. In B, the groups were compared by two-way Anova.

FIG. 3: LINC00518 expression is restricted to melanocytes

Normal skin and melanoma sections were analyzed for MITF and LINC00518 expression by FISH (fluorescence in situ hybridization) using the RNAscope multifluorescent kit. Probes against bacterial RNAs (NEG) were used as negative controls.

FIG. 4: LINC00518 is expressed in melanocytic and mesenchymal melanoma cell lines

Total RNA extracted from melanocytic (MM117; IGR-37; SKMEL28; 501MEL; SKMEL25; M229; M249; M249R; MM011) and mesenchymal (IGR-39; SKMEL25R; M229R; MM029; MM099; MM047) melanoma cell lines was retrotranscribed and analyzed by quantitative PCR. LINC00518 levels were normalized against the levels of three housekeeping genes (TBP, HBMS, RPL13A) to account for variation between cell lines.

FIG. 5: LINC00518 is essential for melanoma cell line proliferation Melanoma cell lines were transfected with LNA gapmers targeting LINC00518 and 72 hours post transfection were counted (A) and harvested for total RNA extraction and quantitative PCR analysis of LINC00518 levels (B). Cell numbers of Gapmer transfected cells were compared to the negative control Gapmer by one-way Anova (Dunn test). LINC00518 negative HEKT cells were taken as a negative control to exclude toxic effects.

FIG. 6: LINC00518 is essential for normal melanoma cell cycle

(A) Proliferation of MM011 or MM047 melanoma cells transfected with a negative control or LINC00518 specific Gapmers was evaluated by Cell Trace Violet Staining. Single cells were gated based on physical parameters and on Cell Trace mean fluorescence value. Cells with higher Cell Trace fluorescence were considered as low proliferative and the percentages of knock down samples were compared to the negative control by one-way Anova (Dunn test). (B) 501me1 melanoma cells were transfected with the indicated Gapmers and analyzed by flow cytometry 72 hours after. Cells were incubated 1 hour and 30 minutes with 5-ethynyl-2′-deoxyuridine (Edu), fixed, permeabilized and stained with fluorescein (FITC) by Click-it reaction. DNA was stained with TOPRO-3 and single cells were gated based on physical parameters, FITC and TOPRO-3 mean fluorescent values. Cells negative for Edu-FITC and TOPRO-3 low were considered as being in G1 phase; cells Edu positive as being in the S phase; cells Edu negative/TOPRO-3 high as G2/M.

FIG. 7: LINC00518 is essential for normal melanoma cell mitosis 501me1 melanoma cells were transfected with Gapmers targeting LINC00518 and analyzed by immunofluorescence 72 hours after. Cells were fixed and incubated with antibodies against tubulin (A), phospho-gamma H2AX (B) and with for DAPI for DNA. Mitotic abnormalities and DNA damage were evaluated by counting the number of multinucleated cells (A) and phospho-gamma H2AX positive cells (B) over the total in at least 3 different fields. Significant differences between CTR and knock down samples were evaluated by one-way Anova (Dunn Test).

FIG. 8: LINC00518 is essential for melanoma survival

(A) Survival of melanoma cells transfected with a negative control or LINC00518 specific Gapmers was evaluated by active caspase 3 staining. Single cells were gated based on physical parameters and active caspase 3 mean fluorescent value. Cells with higher fluorescence were considered as apoptotic. LINC00518 negative HEKT cells were taken as a negative control to exclude non-specific toxic effects. (B) Survival of 501me1 melanoma cells transfected with a negative control or LINC00518 specific Gapmers was evaluated by annexin V/TOPRO-3 stainings. Single cells were gated based on physical parameters and annexin V, TOPRO-3 mean fluorescent values. AnnexinV-/TOPRO-3− cells were considered as viable; AnnexinV+/TOPRO-3- as early apoptotic; AnnexinV+/TOPRO-3+ as late apoptotic; AnnexinV-/TOPRO-3+as necrotic. The percentages of viable knock down cells were compared to the negative control by one-way Anova (Dunn test).

FIG. 9: No evidence for LINC00518 acting via miR-RNAs or via the JAK-STAT pathway in melanoma SKCM TCGA RNA sequencing data were analyzed for LINC00518, MIR199A1, MIR199A2, MIR2166, MRP1 and CDX2 expression. Pairwise Spearman correlation (r) was calculated between LINC00518 and MIR199A1, MIR199A2, MRP1, CDX2 (A) and between MRP1 and MIR199A1, MIR199A2 (B). (C) 501mel cells were transfected with negative control (CTR) or a Gapmer targeting LINC00518 (GAP). Total RNA was extracted 48 hours post transfection and sequenced. Graph shows normalized read counts for LINC00518, MRP1 and CDX2. Significance between CTR and GAP samples was calculated by two-tailed Student t-test. (D) Cervical carcinoma (siHA) and melanoma cell lines (501mel, MM011, MM047) were transfected with negative control (CTR) or LINC00518 specific gapmers (GAP) and total protein extracts were prepared 72 hours post transfection. Samples were analyzed by immunoblot for phosphor-STAT3 on tyrosine 705 using a specific antibody. Vinculin was taken as a loading control.

FIG. 10: Vemurafenib potentiates melanoma cell apoptosis induced by LINC00518 knockdown.

A. LINC00518 expression in public datasets of melanoma cells treated with the BRAF inhibitor Vemurafenib was mined showing its expression was strongly up regulated by 3 days during the acute phase of the response before being down regulated. B. 501Mel cells were treated Vemurafenib and LINC00518 expression was assessed by RT-qPCR showing that was induced from 12 hours to 6 days after treatment. C. 501Mel cells were treated for 3-6 days with Vemurafenib or with DMSO control and then transfected with suboptimal doses of negative control or LINC00518 GapmeR. Activated Caspase 3 was measured by FACS.

FIG. 11: LINC00518 localises to mitochondria and regulates oxidative phosphorylation in melanoma cells. A. Cytoplasmic and mitochondrial RNA fractions were extracted from melanoma cells, retrotranscribed and analyzed by qPCR for the mitochondrial RNAs 16S and SAMMSON and for

LINC00518. LINC00518 negative HEKT cells were taken as a negative control. B. Oxygen consumption rate (OCR) was measured using Agilent Seahorse 48 hours after transfection with a negative control (GAP-CTR) or LINC00518 specific (GAP-518). Gapmer. Basal and Maximal OCR and reserve capacity were calculated on the basis of OCR changes upon oligomycin (oligo), FCCP and Rotenone A (RotA) administration to the cells.

FIG. 12: LINC00518 over-expression increases oxidative phosphorylation and proliferation of melanoma cells. A. 501me1 melanoma cells were infected with a lentiviral vector to induce LINC00518 or GFP expression in a doxycycline-dependent manner. After doxycycline administration, cells were cultured for up to 4 days, total RNA was extracted, retrotranscribed and analyzed by qPCR for LINC00518 expression. Results are expressed as fold change over the sample of untreated cells at time 0. B. 501me1 melanoma cells modified as in A were cultured for 4 days in the presence of doxycycline and analyzed using the Agilent Seahorse. Basal and Maximal OCR and reserve capacity were calculated on the basis of OCR changes upon oligomycin (oligo), FCCP and Rotenone A (RotA) administration to the cells. C. 501me1 melanoma cells untransduced (utd) or modified as in A were seeded at 500 cells/9.6 cm2 and cultured for 10 days in presence of doxycycline. Cells were fixed, stained with crystal violet and colonies counted to estimate cells clonogenic capacity. D. 501me1 melanoma cells modified as in A were seeded at 500 cells/9.6 cm2 and cultured for 10 days in presence or absence of doxycycline. Cells were fixed, stained with crystal violet and the % of the area occupied by the cells was calculated. E. 501me1 melanoma cells modified as in A were seeded at 750 000 cells/56.7 cm2 and cultured for 12 days in presence of doxycycline in non-adherent conditions. Cells pictures were taken every three days and the area occupied by melanospheres was quantified.

FIG. 13: LINC00518 is increased upon MAPK inhibition in vitro and in vivo and sustains metabolic adaptation. A. A375 and 501me1 BRAF mutant melanoma cells were cultured with Vemurafenib, Trametinib or Dabrafenib over 6 days. Samples were taken at the indicated time points, total RNA was extracted, retrotranscribed and analyzed by qPCR for LINC00518 expression. B. LINC00518 RNA levels were quantified from scRNAseq of melanoma patient derived xenotransplants (PDXs) treated with Dabrafenib and Trametinib (GSE116237). C. LINC00518 RNA levels were quantified from RNA sequencing of melanoma patients treated with Durvalumab and Trametinib for 15 days (GSE158403). D. A375 cells were cultured for 6 days with Vemurafenib. Cytoplasmic and mitochondrial RNA fractions were extracted, retrotranscribed and analyzed by qPCR for LINC00518 expression. E. 501me1 melanoma cells were treated with DMSO or Vemurafenib for 6 days and transfected with a negative control (GAP-CTR) or LINC00518 specific (GAP-518) Gapmer during the last 48 hours. Oxygen consumption rate (OCR) was measured using Agilent Seahorse. Basal and Maximal OCR and reserve capacity were calculated on the basis of OCR changes upon oligomycin (oligo), FCCP and Rotenone A (RotA) administration to the cells.

FIG. 14: LINC00518 inhibition synergizes with MAPK pathway targeting to induce apoptosis. 501mel (A) and A375 (B) melanoma cells were treated with DMSO or Vemurafenib for 6 days, stained with Cell Trace Violet and transfected with a negative control (GAP-CTR) or LINC00518 specific (GAP-518) Gapmer during the last 48 hours. Cells were fixed, stained with an anti-active caspase 3 antibody and analyzed by flow cytometry. Single cells were gated based on physical parameters and on Cell Trace mean fluorescence value or Active Caspase 3 positive staining. Cells with higher Cell Trace fluorescence were considered as low proliferative. 501me1 (C) and A375 (D) melanoma cells were treated with DMSO, Vemurafenib or Dabrafenib+ Trametinib (DT) for 6 days and transfected with a negative control (GAP-CTR) or LINC00518 specific (GAP-518) Gapmer during the last 48 hours. Cells were fixed, stained with Crystal violet and the % of the area occupied by the cells was calculated.

FIG. 15: LINC00518 over-expression reduces Vemurafenib anti-proliferative effect. A. 501me1 melanoma cells were infected with a lentiviral vector to induce LINC00518 or GFP expression in a doxycycline-dependent manner. Cells were cultured for 6 days with Doxycyline in the presence or absence of Vemurafenib. Oxygen consumption rate (OCR) was measured using Agilent Seahorse. Basal and Maximal OCR and reserve capacity were calculated on the basis of OCR changes upon oligomycin (oligo),

FCCP and Rotenone A (RotA) administration to the cells. B. 501me1 melanoma cells modified as in A were seeded at 750 000 cells/56.7 cm2 and cultured for 15 days with doxycycline in the presence or absence of Vemurafenib. Cells pictures were taken every three days and the area occupied by melanospheres was quantified.

FIG. 16: LINC00518 promotes mitochondrial fusion. A. 501me1 and A375 cells were transfected with a negative control (GAP-CTR) or L518 specific (GAP-518) GapmeR, cultured on microscope slides and incubated two hours with MitotrackerCMXROS Red and Hoescht to stain mitochondria and DNA respectively. Cells were analyzed by confocal microscopy without fixation keeping the temperature of the chamber at 37° C. B. 501me1 and A375 cells were treated with DMSO or Vemurafenib (1 uM) for three days and transfected with L518 GapmeR (VEM+GAP-518), cultured for a further three days with DMSO or Vemurafenib and analyzed by confocal microscopy as in A.

### DETAILED DESCRIPTION OF THE INVENTION

The present invention provides a new strategy for the treatment of melanoma. This strategy is based on the use of antisense oligonucleotide targeting LINC00518. The antisense oligonucleotide targeting LINC00518 both increases melanoma cancer cell apoptosis and decreases melanoma cancer cell proliferation, the melanoma cancer cells being either the melanocytic or undifferentiated/mesenchymal phenotype and irrespective of their mutational status (e.g., BRAF or NRAS mutants). The impact on the two types of cells is important because melanocytic cells would be involved in proliferative aspect of melanoma whereas mesenchymal cells would be involved in the invasive and therapy resistance aspects.

When considering that the survival of patients having melanoma metastasis is dramatically lower, it is key to have an effect on both types of cells. The antisense oligonucleotide targeting LINC00518 also affects resistant cells, in particular those resistant to BRAF inhibitors. As the occurrence of resistance is a problem in the treatment of melanoma, this is an important advantage. Accordingly, the present invention relates to an antisense oligonucleotide as defined herein for use for preventing, decreasing or delaying the appearance of a resistance to a targeted therapy.

Targeting of LINC00518 is of particular interest because this long non-coding RNA (IncRNA) is predominantly expressed in melanoma. In addition, the level of expression is correlated with the melanoma stage and then to the melanoma aggressiveness.

Up to now, the role of LINC00518 has been studied in the context of breast cancer (Chang et al, 2018, Cell Physiol Biochem, 48, 16-28; Wang et al, 2019, BBA— Molecular Basis of Disease, 1865, 708-723).

More particularly, Chang et al studied effect of LINC00518 on the resistance of breast cancer cells. They observed an increased expression of LINC00518 in ADR-resistant MCF-7 cell lines in comparison to MCF-7 and that LINC00518 knock-out (KO) enhances chemosensitivity. The authors conclude that LINC00518 would inhibit miR-199a expression and that miR-199a is known as a tumor suppressor in some particular cancers. Indeed, the inhibition of miR-199a would abrogate the effect of LINC00518 (KO). However, in the context of melanoma, Zhou et al (2014, Int J Clin Exp Pathol, 7, 7182-7190) referring to observations from another study (Pencheva et al, 2012, Cell, 151, 1068-1082) suggest that miR-199a-5p and miR-199a-3p may drive metastasis and angiogenesis in the context of melanoma. Indeed, Pencheva et al exhibited some results showing that overexpression of both miR-199a-3p and miR-199a-5p was sufficient to robustly increase lung metastatic colonization and conversely, that the individual inhibition of miR-199a-3p or miR-199a-5p in a metastatic cell (MeWo-LM2.3 cell line) significantly suppressed metastatic colonization (see page 2, last paragraph). Therefore, the observations made in breast cancer do not seem relevant in the context of melanoma.

The same remark will apply for Wang et al. The authors note that down-regulation of LINC00518 inhibits proliferation, invasion, migration and EMT of breast cancer epithelial cells while enhancing apoptosis. The mechanism described by the authors would be that LINC00518 inhibition up-regulates CDX2 expression. However, CDX2 is expressed in tissues derived from mesoderm, and not in tissues derived from the neural crest or neuroectoderm. Then, CDX2 is not expressed in cutaneous or uveal melanoma.

A very recent article of Luan et al (2019, Cell Death and Disease, 10:855) suggests that LINC00518 would be involved in the promotion of metastasis of malignant melanoma via miR-204-5p/AP1S2 axis. The authors noted that LINC00518 is mainly localized in cytoplasm and acts by sponging miR-204-5p, an event that occurs in the cytoplasm. They showed that a shRNA or siRNA targeting LINC00518 decreases invasive and migratory ability but does not have any effect on the growth and apoptosis of melanoma cells. It is known that shRNA and siRNA are tools that are adapted to target IncRNA in cytoplasm as discussed in Lennox and Behlke (2016, J Rare Dis Res Treat, 1, 66-70) because RNAi-mediated RNA degradation occurs in cytoplasm. Luan et al do not provide any information about the sequence of shRNA and siRNA used in the experiments, nor of their efficiency in inducing LINC00518 knockdown nor of their selectivity.

Surprisingly, the inventors discovered that an antisense oligonucleotide targeting LINC00518 has new effects on melanoma cells. Indeed, the antisense oligonucleotide targeting LINC00518 is capable of both decreasing the proliferation of melanoma cells and decreasing cell survival by increasing apoptosis. They showed the specificty of this effect as no apoptosis is seen upon antisense oligonucleotide targeting of LINC00518 in cells where it is not expressed. Then, these effects are unpredictable and advantageous for the treatment of melanoma because they induce a decrease of tumor growth and not only impair invasive and migratory ability. The approach of the inventors based on the use of an antisense oligonucleotide is original and not suggested by the prior art because it was known that the antisense oligonucleotide uses the RNase H for degrading the target and the RNase H is most abundant in the nucleus and in the mitochondria.

The inventors surprisingly identified the importance of LINC00518 in the mitochondrial metabolism and the importance to inhibit LINC00518 in the mitochondria. An antisense oligonucleotide targeting LINC00518 is appropriate for this inhibition in the mitochondria, which would not be the case for an antisense RNA.

Finally, the inventors showed a synergistic effect of the combination of an antisense oligonucleotide targeting LINC00518 and a drug acting on MAPK pathway, especially to induce apoptosis. More specifically, the drug acting on MAPK pathway can be a B-Raf inhibitor such as Vemurafenib or Dabrafenib and/or a MEK inhibitor such as Trametinib. This synergistic effect allows to contemplate the use of lower amount of drug acting on MAPK pathway. In particular, a sub-therapeutic amount can be used for the treatment of melanoma. It is of interest in order to decrease the side effects that can be associated with a drug acting on MAPK pathway.

Therefore, the present invention relates to an antisense oligonucleotide targeting LINC00518 or inhibiting the expression of LINC00518, a pharmaceutical composition comprising an antisense oligonucleotide targeting LINC00518 or inhibiting the expression of LINC00518, the antisense oligonucleotide or pharmaceutical composition for use for the treatment of melanoma, the use of the antisense oligonucleotide or pharmaceutical composition for the manufacture of a medicament for the treatment of melanoma, and a method of treatment of a subject having a melanoma comprising administering a therapeutically effective amount of the antisense oligonucleotide or pharmaceutical composition.

### Definition

LINC00518 refers to the Long Intergenic Non-Protein Coding RNA 518. It is disclosed in the GeneCards database under ID GC06M010428, in HGNC database under ID 28626, in Gene database under ID 221718, Ensembl database ID ENSG00000183674 and in the Genbank database under NR_027793. It is also called C6orf218. LINC00518 gene comprises 4 exons and encodes 5 splicing variants, called isoforms 1, 2, 3, 5 and 6.

| LINC00518 | SEQ ID NO: |
|---|---|
| Isoform 1 | 1 |
| Isoform 2 | 2 |
| Isoform 3 | 3 |
| Isoform 5 | 4 |
| Isoform 6 | 5 |
| 1^{st} exon of isoform 1 (exon 1) | 6 |
| 1^{st} intron of isoform 1 | 7 |
| 2^{nd} exon of isoform 1 (exon 4) | 8 |
| 1^{st} exon of isoform 2 (exon 1) | 9 |
| 1^{st} intron of isoform 2 | 10 |
| 2^{nd} exon of isoform 2 (exon 2) | 11 |
| 2^{nd} intron of isoform 2 | 12 |
| 3^{rd} exon of isoform 2 (exon 4) | 13 |
| 1^{st} exon of isoform 3 (exon 1) | 14 |
| 1^{st} intron of isoform 3 | 15 |
| 2^{nd} exon of isoform 3 (exon 2) | 16 |
| 2^{nd} intron of isoform 3 | 17 |
| 3^{rd} exon of isoform 3 (exon 4) | 18 |
| 1^{st} exon of isoform 5 (exon 1) | 19 |
| 1^{st} intron of isoform 5 | 20 |
| 2^{nd} exon of isoform 5 (exon 2) | 21 |
| 2^{nd} intron of isoform 5 | 22 |
| 3^{rd} exon of isoform 5 (exon 3) | 23 |
| 3^{rd} intron of isoform 5 | 24 |
| 4^{th} exon of isoform 5 (exon 4) | 25 |
| 1^{st} exon of isoform 6 (exon 1) | 26 |
| 1^{st} intron of isoform 6 | 27 |
| 2^{nd} exon of isoform 6 (exon 4) | 28 |

As used herein, “treating,” “treatment” or “therapy” is an approach for obtaining beneficial or desired clinical results. This includes: increase the alleviation of symptoms, the reduction of inflammation, the inhibition of cancer cell growth, and/or the reduction of tumor size. In some embodiments, the term treatment refers to the inhibition or reduction of cancer cell proliferation in a subject having cancer. Furthermore, these terms are intended to encompass curing as well as ameliorating at least one symptom of the condition or disease. For example, in the case of cancer, a response to treatment includes a reduction in cachexia, increase in survival time, elongation in time to tumor progression, reduction in tumor mass, reduction in tumor burden and/or a prolongation in time to tumor metastasis, time to tumor recurrence, tumor response, complete response, partial response, stable disease, progressive disease, progression free survival, overall survival, each as measured by standards set by the National Cancer Institute and the U.S. Food and Drug Administration for the approval of new drugs. See Johnson et al. (2003) J. Clin. Oncol. 21(7): 1404-1411.

As used herein, the term “subject” or “patient” refers to an animal, preferably to a mammal, even more preferably to a human, including adult or child.

The terms “kit”, “product” or “combined preparation”, as used herein, define especially a “kit-of-parts” in the sense that the combination partners (a) and (b), as defined above can be dosed independently or by use of different fixed combinations with distinct amounts of the combination partners (a) and (b), i.e. simultaneously or at different time points. The components of the kit-of-parts can then, e.g., be administered simultaneously or chronologically staggered, that is at different time points and with equal or different time intervals for any part of the kit-of-parts. The ratio of the total amounts of the combination partner (a) to the combination partner (b), to be administered in the combined preparation can be varied. The combination partners (a) and (b) can be administered by the same route or by different routes.

By “effective amount” or “therapeutically effective amount”, it is meant the quantity of the pharmaceutical composition, kit, product and combined preparation of the invention which prevents, removes or reduces the deleterious effects of cancer in mammals, including humans, alone or in combination with the other active ingredients of the pharmaceutical composition, kit, product or combined preparation. It is understood that the administered dose may be adapted by those skilled in the art according to the patient, the pathology, the mode of administration, etc.

As used herein, the term “sub-therapeutic amount” or “sub-therapeutic dose” refers to a dosage which is less than that dosage which would produce a therapeutic result in the subject if administered in the absence of the other agent. For instance, “sub-therapeutic amount” or “sub-therapeutic dose” can refer to a dosage which is decreased by 25, 50, 70, 80 or 90% in comparison to the therapeutically effective amount, especially the conventional therapeutic dosage for the same indication and the same administration route when used alone. The conventional therapeutic dosages are those acknowledged by the drug approvals agencies (e.g., FDA or EMEA).

By “a synergistic effect” is intended to refer to an effect for decreasing melanoma cell survival, especially for increasing melanoma cells apoptosis which is more than the sum of the effects of each molecule alone.

Antisense Oligonucleotide Targeting LINC00518

The present invention relates to an antisense oligonucleotide targeting LINC00518 or to an ASO inhibiting expression of LINC00518.

As used herein, an “ASO” refers to a modified single-stranded oligonucleotide comprising at least one region which is complementary to a target nucleic acid. ASOs are designed and commonly used to modulate the expression of their target nucleic acid, notably to knock down their target. The precise targeting of a specific nucleic acid, on which the selectivity of a knockdown strategy depends, relates to a balance between oligonucleotide length and complementarity rate toward the defined target. In addition, chemical modifications are generally required to confer an improvement in single-stranded oligonucleotide stability within cells, especially towards digestion by nucleases. Indeed, unmodified single-stranded oligonucleotides are too instable to use in cells. Notably, it is well known by the one skilled in the art that the nuclease resistance can be dramatically improved by modifying internucleotide linkage, e.g., by substituting phosphodiester bonds by phosphorothioate (PS) linkage. Furthermore, other chemical modifications can improve potency and selectivity of ASO by increasing binding affinity of ASOs for their target.

The ASO according to the invention is a single-stranded oligonucleotide comprising deoxyribonucleotides and/or ribonucleotides. In a first embodiment, the ASO according to the invention comprises ribonucleotides and deoxyribonucleotides, i. e. DNA or DNA-like nucleotides. In a second embodiment, the ASO according to the invention comprises only deoxyribonucleotides, i. e. DNA or DNA-like nucleotides. In a third embodiment, the ASO according to the invention comprises only ribonucleotides, i.e. RNA or RNA-like nucleotides.

According to their composition in nucleotides, i. e. ribonucleotides and deoxyribonucleotides or ribonucleotides exclusively, the ASO according to the invention can inhibit the expression of its target nucleic acid via different ways.

In a preferred embodiment, the ASO acts via RNase H mediated degradation. RNase H is a cellular enzyme which recognizes the duplex between DNA and RNA, and enzymatically cleaves the RNA molecule. Thus, ASO comprises a region that comprises DNA or DNA-like nucleotides complementary to the targeted LINC00518 which is responsible for RNAse H recruitment that leads to subsequent target nucleic acid cleavage.

Alternatively, the ASO according to the invention can inhibit the expression of LINC00518 by inhibiting the translation of LINC00518. In another particular embodiment, the ASO according to the invention can inhibit the formation of mature RNAs of LINC00518 by modulating the splicing of the pre RNAs of LINC00518.

The ASO according to the invention has an overall sequence length of at least 10 nucleotides, preferably at least 12 nucleotides, more preferably at least 16 nucleotides. In a preferred aspect, the ASO according to the invention has an overall sequence length of 10 to 30 nucleotides, more preferably of 12 to 30, 12 to 26, 13 to 24, 14 to 22, 14 to 17 or 16 to 18 nucleotides.

As the target of the ASO of the invention is LINC00518, the ASO comprises a nucleotide sequence which is complementary to LINC00518, in particular to a specific region of the LINC00518 sequence, in particular of any of the sequences of SEQ ID Nos: 1-28.

The ASO according to the invention comprises a contiguous nucleotide sequence of 10 to 30 nucleotides in length that is complementary to a specific portion of LINC00518, in particular of any of the sequences of SEQ ID Nos: 1-28. Preferably, the length of the ASO contiguous sequence is 12 to 30 contiguous nucleotides, alternatively 12 to 26, 13 to 24, 14 to 22, 14 to 17 or 16 contiguous nucleotides in length that is complementary to a specific portion of LINC00518, in particular of any of the sequences of SEQ ID Nos: 1-28.

According to the invention, the ASO contiguous nucleotide sequence is at least 90, 95, 96, 97, 98, 99 or 100 percent complementary to a specific region of LINC00518, in particular of any of the sequences of SEQ ID Nos: 1-28. Optionally, the ASO can be complementary to any region of LINC00518, for instance in an exon of LINC00518, in particular as disclosed in any of the sequences of SEQ ID Nos: 6, 8, 9, 11, 13, 14, 16, 18, 19, 21, 23, 25, 26 and 28, or an intron of LINC00518, in particular as disclosed in any of the sequences of SEQ ID Nos: 7, 10, 12, 15, 17, 20, 22, 24 and 27. More specifically, the ASO contiguous nucleotide sequence is at least 90, 95, 96, 97, 98, 99 or 100 percent complementary to exon 1 of LINC00518 (in particular as disclosed in any of the sequences of SEQ ID Nos: 6, 9, 14, 19 and 26), exon 2 of LINC00518 (in particular as disclosed in any of the sequences of SEQ ID Nos: 11, 16 and 21), exon 3 of LINC00518 (in particular as disclosed in the sequence of SEQ ID No: 23) or exon 4 of LINC00518 (in particular as disclosed in any of the sequences of SEQ ID Nos: 8, 13, 18, 25 and 28). In a first aspect, the ASO contiguous nucleotide sequence is at least 90, 95, 96, 97, 98, 99 or 100 percent complementary to exon 1, in particular as disclosed in any of the sequences of SEQ ID Nos: 6, 9, 14, 19 and 26. In a second aspect, the ASO contiguous nucleotide sequence is at least 90, 95, 96, 97, 98, 99 or 100 percent complementary to exon 2, in particular as disclosed in any of the sequences of SEQ ID Nos: 11, 16 and 21. In a third aspect, the ASO contiguous nucleotide sequence is at least 90, 95, 96, 97, 98, 99 or 100 percent complementary to exon 3, in particular as disclosed in the sequence of SEQ ID No: 23. In a fourth aspect, the ASO contiguous nucleotide sequence is at least 90, 95, 96, 97, 98, 99 or 100 percent complementary to exon 4, in particular as disclosed in any of the sequences of SEQ ID Nos: 8, 13, 18, 25 and 28. In a preferred aspect, the ASO contiguous nucleotide sequence is at least 90, 95, 96, 97, 98, 99 or 100 percent complementary to exon 1 or exon 4.

Optionally, the ASO contiguous nucleotide sequence may comprise 0, 1, 2 or 3 mismatches in complementarity toward the targeted specific region of LINC00518. Preferably, the complementarity between the ASO contiguous nucleotide sequence and LINC00518 sequence comprises 0 to 2, 0 or 1, or more preferably 0 mismatch.

In particular embodiments, the ASO according to the invention comprises a contiguous nucleotide sequence of 10 to 30 nucleotides in length that is at least 90, 95, 96, 97, 98, 99 or 100 percent complementary to a specific region of the LINC00518 sequence, in particular as disclosed in any of the sequences of SEQ ID Nos: 6, 8, 9, 11, 13, 14, 16, 18, 19, 21, 23, 25, 26 and 28; preferably exon 1, in particular as disclosed in any of the sequences of SEQ ID Nos: 6, 9, 14, 19 and 26, exon 2, in particular as disclosed in any of the sequences of SEQ ID Nos: 11, 16 and 21, exon 3, in particular as disclosed in the sequence of SEQ ID No: 23 or exon 4, in particular as disclosed in any of the sequences of SEQ ID Nos: 8, 13, 18, 25 and 28. In preferred embodiments, the ASO according to the invention comprises a contiguous nucleotide sequence of 12 to 30 nucleotides in length, that is at least 90, 95, 96, 97, 98, 99 or 100 percent complementary to exon 1, in particular as disclosed in any of the sequences of SEQ ID Nos: 6, 9, 14, 19 and 26, exon 2, in particular as disclosed in any of the sequences of SEQ ID Nos: 11, 16 and 21, exon 3, in particular as disclosed in the sequence of SEQ ID No: 23 or exon 4, in particular as disclosed in any of the sequences of SEQ ID Nos: 8, 13, 18, 25 and 28.

The ASO according to the invention comprises chemical modifications that confer an improved stability of single-stranded oligonucleotides within cells, in particular modifications relative to internucleotide linkages.

In a preferred aspect, the ASO according to the invention comprises phosphorothioate linkages in place of the phosphodiester bonds. The phosphorothioate linkages are preferably localized at the ends of the ASO. More preferably, the ASO according to the invention comprises at least 10, 11, 12, 13, 14 or 15 phosphorothioate linkages in place of the phosphodiester bonds. In a particular aspect, all the internucleotide linkage of ASO are phosphorothioate linkages.

In a preferred aspect, the ASO according to the invention comprises chemical modifications allowing an increase in binding affinity of oligonucleotides for their target nucleic acid. Such nucleotide modifications can be, but are not limited to, the addition on ribose of group such as 2′-O-methyl (2′-O-Me), 2′-fluoro (2′-F), 2′-O-methoxyethyl (MOE), the introduction of methylene bridge between the 2′ and 4′ position of the ribose which define the “locked” nucleic acids (LNAs), or the introduction of constrained ethyl (cEt) bridged nucleic acid (BNA). Besides, phosphorodiamidate morpholinos (PMOs) represent another modification that enhances metabolic stability and affinity for ribonucleotides by replacing the sugar and backbone with a morpholino ring system. All these modifications are well known to the one skilled in the art and also contribute to enhance the oligonucleotide stability (see Watts et al. J Pathol. 2012 January; 226(2): 365-379; Seth et al. J Clin Invest. 2019; 129(3):915-925).

In particular embodiments, the ASO according to the invention comprises 2′-O-Me, 2′-F, MOE, LNA, cET or PMO modified nucleotides in the contiguous nucleotide sequence, preferably 2′-O-Me, 2′-F, MOE, or LNA modified nucleotides, more preferably MOE and/or LNA modified nucleotides.

In a preferred embodiment, the ASO according to the invention comprises at least 1, 2, 3, 4, 5, 6, 7, 8, 9, 10, 11, or 12 LNA modified nucleotides. In another preferred embodiment, the ASO according to the invention comprises at least 1, 2, 3, 4, 5, 6, 7, 8, 9, 10, 11, or 12 MOE modified nucleotides. In a preferred aspect, the ASO according to the invention is capable of inhibiting the expression of LINC00518 in the nucleus and in the cytoplasm. More particularly, the ASO is capable of decreasing the expression of LINC00518 by 10, 20, 30, 40, 50, 60, 70, 80, 90, 95 or 99% in comparison of its expression in absence of the ASO.

In one particular aspect, the ASO according to the invention is capable of inhibiting the expression of one or more isoforms of LINC000518, especially isoforms selected in the group consisting of 1, 2, 3, 5 and 6. Optionally, it is able to inhibit the expression of at least 2, 3, 4 or 5 isoforms of LINC00518. In a particular aspect, it is able to inhibit the expression of isoform 1, 5 or 6 or combinations thereof such as isoform 1 and isoform 5, isoform 1 and isoform 6, or isoform 5 and isoform 6. In another very particular aspect, it is able to inhibit the expression of isoforms 1, 5 and 6. In a very particular aspect, it is able to inhibit the expression of isoforms 1, 2, 3, 5 and 6.

Gapmers

In a preferred aspect, the ASO of the invention may be a gapmer, also termed gapmer oligonucleotide, antisense gapmer or gapmer designs. Classically, a gapmer comprises at least three distinct structural regions, namely a 5′ flank region (F), a gap region (G) and a 3′ flank region (F′). Besides, the F and F′ regions are composed of modified ribonucleotides (RNA*) whereas the G region is composed of deoxyribonucleotides, i. e. DNA or DNA-like nucleotides.

The antisense gapmers are commonly used to inhibit a target nucleic acid via RNase H mediated degradation. Thus, the G region that comprises DNA or DNA-like nucleotides is responsible for RNAse H recruitment that leads to subsequent target nucleic acid cleavage. In contrast, the F and F′ regions comprise contiguous ribonucleotide sequence that are complementary to a target nucleic acid, i. e. two distinct regions of their target, and are thus responsible for the binding specificity to this target.

The antisense gapmer according to the invention has an overall sequence length of at least 10 contiguous nucleotides, preferably at least 12 contiguous nucleotides, more preferably at least 16 contiguous nucleotides, that is complementary to a specific portion of LINC00518, in particular of any of the sequences of SEQ ID Nos: 1-28.

In a preferred aspect, the antisense gapmer according to the invention has an overall sequence length of 10 to 30 contiguous nucleotides, more preferably of 12 to 30, 12 to 26, 13 to 24, 14 to 22, 14 to 17 or 16 to 18 contiguous nucleotides, that is complementary to a specific portion of LINC00518, in particular of any of the sequences of SEQ ID Nos: 1-28.

According to the invention, the antisense gapmer contiguous nucleotide sequence is at least 90, 95, 96, 97, 98, 99 or 100 percent complementary to a specific region of LINC00518, in particular of any of the sequences of SEQ ID Nos: 1-28. Optionally, the antisense gapmer can be complementary to any region of LINC00518, for instance in an exon of LINC00518, in particular as disclosed in any of the sequences of SEQ ID Nos: 6, 8, 9, 11, 13, 14, 16, 18, 19, 21, 23, 25, 26 and 28, or an intron of LINC00518, in particular as disclosed in any of the sequences of SEQ ID Nos: 7, 10, 12, 15, 17, 20, 22, 24 and 27. More specifically, the antisense gapmer contiguous nucleotide sequence is at least 90, 95, 96, 97, 98, 99 or 100 percent complementary to exon 1 of LINC00518 (in particular as disclosed in any of the sequences of SEQ ID Nos: 6, 9, 14, 19 and 26), exon 2 of LINC00518 (in particular as disclosed in any of the sequences of SEQ ID Nos: 11, 16 and 21), exon 3 of LINC00518 (in particular as disclosed in the sequence of SEQ ID No: 23) or exon 4 of LINC00518 (in particular as disclosed in any of the sequences of SEQ ID Nos: 8, 13, 18, 25 and 28). In a first aspect, the contiguous nucleotide sequence of the gapmer is at least 90, 95, 96, 97, 98, 99 or 100 percent complementary to exon 1, in particular as disclosed in any of the sequences of SEQ ID Nos: 6, 9, 14, 19 and 26. In a second aspect, the contiguous nucleotide sequence of the gapmer is at least 90, 95, 96, 97, 98, 99 or 100 percent complementary to exon 2, in particular as disclosed in any of the sequences of SEQ ID Nos: 11, 16 and 21. In a third aspect, the contiguous nucleotide sequence of the gapmer is at least 90, 95, 96, 97, 98, 99 or 100 percent complementary to exon 3, in particular as disclosed in the sequence of SEQ ID No: 23. In a fourth aspect, the contiguous nucleotide sequence of the gapmer is at least 90, 95, 96, 97, 98, 99 or 100 percent complementary to exon 4, in particular as disclosed in any of the sequences of SEQ ID Nos: 8, 13, 18, 25 and 28. In a preferred aspect, the contiguous nucleotide sequence of the gapmer is at least 90, 95, 96, 97, 98, 99 or 100 percent complementary to exon 1 or exon 4.

Optionally, the contiguous nucleotide sequence of the gapmer may comprise 0, 1, 2 or 3 mismatches in complementarity toward the targeted specific region of LINC00518. Preferably, the complementarity between the gapmer nucleotide sequence and LINC00518 sequence comprises 0 to 2, 0 or 1, or more preferably 0 mismatch.

In particular embodiments, the gapmer according to the invention comprises a contiguous nucleotide sequence of 10 to 30 nucleotides in length that is at least 90, 95, 96, 97, 98, 99 or 100 percent complementary to a specific region of the LINC00518 sequence, in particular as disclosed in any of the sequences of SEQ ID Nos: 6, 8, 9, 11, 13, 14, 16, 18, 19, 21, 23, 25, 26 and 28; preferably exon 1, in particular as disclosed in any of the sequences of SEQ ID Nos: 6, 9, 14, 19 and 26, exon 2, in particular as disclosed in any of the sequences of SEQ ID Nos: 11, 16 and 21, exon 3, in particular as disclosed in the sequence of SEQ ID No: 23 or exon 4, in particular as disclosed in any of the sequences of SEQ ID Nos: 8, 13, 18, 25 and 28. In preferred embodiments, the gapmer according to the invention comprises a contiguous nucleotide sequence of 12 to 30 nucleotides in length, that is at least 90, 95, 96, 97, 98, 99 or 100 percent complementary to exon 1, in particular as disclosed in any of the sequences of SEQ ID Nos: 6, 9, 14, 19 and 26, exon 2, in particular as disclosed in any of the sequences of SEQ ID Nos: 11, 16 and 21, exon 3, in particular as disclosed in the sequence of SEQ ID No: 23 or exon 4, in particular as disclosed in any of the sequences of SEQ ID Nos: 8, 13, 18, 25 and 28.

In particular embodiments, the gapmer according to the invention consists or comprises a contiguous nucleotide sequence that corresponds to the following classical gapmer formula:

5′−F(RNA*)−G(DNAor DNA-like)−F′(RNA*)−3′

According to these embodiments of the invention, the F-G-F′ contiguous nucleotide sequence of the gapmer has an overall sequence length of at least 10 contiguous nucleotides, preferably at least 12 contiguous nucleotides, more preferably of 10 to 30 contiguous nucleotides, 12 to 30, 12 to 26, 13 to 24, 14 to 22, 14 to 17 or 16 to 18 contiguous nucleotides.

In a preferred embodiment, the ASO of the invention consists of or comprises a gapmer of formula 5′-F-G-F-3′, where region F and F′ region independently comprise or consist of 1 to 8 contiguous ribonucleotides, preferably 2 to 6 or 3 to 4 contiguous ribonucleotides, and G region comprises or consists of 6 to 16 deoxyribonucleotides, preferably 6 to 14, 6 to 12, 6 to 10, 6 to 8, 10 to 15, 10 to 14, or 11 to 15 contiguous deoxyribonucleotides.

As for the other ASOs, gapmer nucleotides are necessarily modified to confer an improved stability to the oligonucleotide within cells. Such modifications are notably relative to the introduction of internucleotides linkages. For instance, it is notably known to the one skilled in the art that the substitution of phosphodiester bonds by phosphorothioate PS linkages.

In a preferred aspect, the antisense gapmer according to the invention comprises phosphorothioate linkages in place of the phosphodiester bonds. The phosphorothioate linkages are preferably localized at the ends of the gapmer. More preferably, the antisense gapmer according to the invention comprises at least 10, 11, 12, 13, 14 or 15 phosphorothioate linkages in place of the phosphodiester bonds. In a particular aspect, all the internucleotide linkage of the gapmer are phosphorothioate linkages.

The F and F′ regions usually comprise modified ribonucleotides that enhance the gapmer binding affinity to its target nucleic acid and thus ensure a better selectivity for the knockdown strategy. In particular embodiments, the antisense gapmer according to the invention comprises 2′-O-Me, 2′-F, MOE, LNA, cET or PMO modified ribonucleotides in the F and F′ regions, preferably 2′-O-Me, 2′-F, MOE, or LNA modified nucleotides, more preferably MOE and/or LNA modified ribonucleotides.

In an aspect of the invention the antisense gapmer consists of or comprises a gapmer of formula 5′-F-G-F′-3′, where region F and F′ independently comprise or consist of 1 to 8, preferably 2 to 6 or 3 to 4 2′ sugar modified nucleotides, wherein there is at least one 2′ sugar modified nucleotide positioned at the 3′ end of region F (adjacent to a deoxynucleotide of region G), and at least one 2′sugar modified nucleoside positioned at the 5′ end of region F′ (positioned adjacent to a deoxynucleotide of region G), and G is a region between 6 and 16 nucleosides which are capable of recruiting RNaseH, preferably a region of 6 to 16, 10 to 15, 10 to 14, such as 11 to 15, or 13 to 15 contiguous deoxynucleotides.

In a preferred embodiment, the antisense gapmer according to the invention comprises at least 1, 2, 3, 4, 5, 6, 7, 8, 9, 10, 11, or 12 LNA modified nucleotides. In another preferred embodiment, the antisense gapmer according to the invention comprises at least 1, 2, 3, 4, 5, 6, 7, 8, 9, 10, 11, or 12 MOE modified nucleotides.

In a preferred aspect, the antisense gapmer according to the invention is capable of inhibiting the expression of LINC00518 in the nucleus, optionally in the nucleus and in the cytoplasm. More particularly, the antisense gapmer is capable of decreasing the expression of LINC00518 by at least 10, 20, 30, 40, 50, 60, 70, 80, 90 or 100% in comparison of its expression in absence of the gapmer.

In certain embodiments, the antisense gapmer according to the invention can be an LNA gapmer, a MOE gapmer, a mixed wing gapmer or an alternating flank gapmer.

In a particular aspect, the gapmer is a LNA gapmer. The term “LNA gapmer” refers to a gapmer oligonucleotide wherein at least one of the affinity enhancing modified nucleotides is an LNA nucleotide, i. e. a nucleotide comprising a methylene bridge between the 2′ and 4′ position of the ribose.

In a particular aspect, the gapmer is a MOE gapmer. The term “MOE gapmer” refers to a gapmer wherein at least one of the affinity enhancing modified nucleotides is an MOE nucleotide, i. e. a nucleotide comprising the addition of a group methoxyethyl (MOE) group at the 2′-O position.

In particular embodiments, the gapmer according to the invention consists or comprises a contiguous nucleotide sequence that corresponds to variant of the classical gapmer formula. Indeed, gapmer of the invention can be a headmer, a tailmer, a mixed wing gapmer, an alternative flank gapmer, a gap-breaker gapmer (also called gap-dispupted gapmer) or comprise additional (D and D′) regions.

The terms “headmers” and “tailmers” refers to gapmer oligonucleotides capable of recruiting RNase H where one of the flank regions is missing, i.e., where only one of the ends of the oligonucleotide comprises affinity enhancing modified ribonucleotides. For headmers, the 3′ flank is missing {i.e., the 5′ flank comprises affinity enhancing modified nucleosides) and for tailmers, the 5′ flank is missing {i.e., the 3′ flank comprises affinity enhancing modified nucleosides). In particular embodiments, the gapmer according to the invention consists or comprises a contiguous nucleotide sequence that corresponds to the following headmer (i) or (ii) tailmer formulas:

5′−F(RNA*)−G(DNAor DNA-like)−3′ (i)

5′−G(DNAor DNA-like)—F′(RNA*)−3′ (ii)

The terms “mixed wing gapmer” refers to a LNA gapmer wherein one or both of region F and F′ comprise a 2′ substituted nucleotide, such as a 2′ substituted nucleotide independently selected from the group consisting of 2′-O-alkyl-RNA units, 2′-O-methyl-RNA units, 2′-amino-DNA units, 2′-fluoro-DNA units, 2′-alkoxy-RNA, MOE units, arabino nucleic acid (ANA) units and 2′-fluoro-ANA units, such as MOE nucleotides. In some embodiments, wherein at least one of region F and F′, or both region F and F′ comprise at least one LNA nucleotide, the remaining nucleotides of region F and F′ are independently selected from the group consisting of MOE and LNA. In some embodiments, wherein at least one of region F and F′, or both region F and F′ comprise at least two LNA nucleotides, the remaining nucleotides of region F and F′ are independently selected from the group consisting of MOE and LNA. In some mixed wing embodiments, one or both of region F and F′ may further comprise one or more deoxynucleotides. Some mixed wing gapmer designs are disclosed in WO2008/049085 and WO2012/109395.

In some gapmers, flank regions F and F′ may comprise both LNA and deoxynucleotides.

The terms “alternative flank gapmer” refers to a gapmer that comprise an alternating motif of LNA-DNA-LNA nucleotides. Alternative flank gapmers are thus LNA gapmer oligonucleotides where at least one of the flanks (F or F′) comprises deoxynucleotides in addition to the LNA nucleotide(s). In some embodiments, at least one of region F or F′, or both region F and F′, comprise both LNA nucleotides and deoxynucleotides. In such embodiments, the flanking region F or F′, or both F and F′ comprise at least three nucleotides, wherein the 5′ and 3′ most nucleotides of the F and/or F′ region are LNA nucleotides. Besides, an alternating flank region may comprise up to 3 contiguous deoxynucleotides, such as 1 to 2 or 1 or 2 or 3 contiguous deoxynucleotides.

The terms “gap breaker gapmer” or “gap-disrupted gapmer” refers to a gapmer wherein the G region comprise at least one 3′ endo modified nucleotides. There are numerous reports of the insertion of a modified nucleoside which confers a 3′ endo conformation into the gap region of gapmers, whilst retaining some RNase H recruitment capacity, see for example WO2013/022984.

Importantly, gap-breaker gapmer retain sufficient region of deoxynucleotides within the gap region to allow for RNase H recruitment. The ability of gap-breaker gapmers to recruit RNase H is typically sequence or even compound specific: see Rukov et al. 2015 Nucl. Acids Res. Vol. 43 pp. 8476-8487, which discloses gap-breaker gapmers recruiting RNase H, which in some instances provide a more specific cleavage of the target RNA.

In addition, modified nucleotides used within the gap region of gap-breaker oligonucleotides may for example be modified nucleosides which confer a 3′endo conformation, such as 2′-O-methyl (OMe) or MOE nucleotides, or even beta-D LNA nucleotides (the bridge between 2′ and 4′ of the ribose sugar ring of a nucleotide is in beta conformation), such as beta-D-oxy LNA or ScET nucleosides.

Some gap region of gap-breaker or gap-disrupted gapmers have a deoxynucleotide at the 5′ end of the gap (adjacent to the 3′ ribonucleotide of region F), and a deoxynucleotide at the 3′ end of the gap (adjacent to the 5′ ribonucleotide of region F′). Gapmers which comprise a disrupted gap typically retain a region of at least 3 or 4 contiguous deoxynucleotides at either the 5′ end or 3′ end of the gap region.

In some embodiments, region G of a gap disrupted gapmer comprises at least 6 deoxynucleotides, such as 6, 7, 8, 9, 10, 1 1, 12, 13, 14, 15 or 16 deoxynucleotides. Also, the deoxynucleotides may be contiguous or may optionally be interspersed with one or more modified nucleotides, with the proviso that the gap region G is capable of mediating an effective RNase H recruitment.

The gapmer according to the invention may in some embodiments comprise or consist of the contiguous nucleotide sequence of the classical gapmer formula, i.e. F-G-F′, and further comprising 5′ and/or 3′ nucleotides. The further 5′ and/or 3′ nucleotides may or may not be fully complementary to the target nucleic acid. Such further 5′ and/or 3′ nucleotides may be referred to as region D′ and D″ herein.

The addition of region D′ or D″ may be used for the purpose of joining the contiguous nucleotide sequence of the gapmer to a conjugate moiety or another functional group. When used for joining the gapmer sequence with a conjugate moiety, one peripheral region, i. e. D′ and/or D″, can serve as a biocleavable linker (described below). Alternatively, it may be used to provide exonuclease protection or for ease of synthesis or manufacture.

Region D′ and D″ can be attached to the 5′ end of region F (i), the 3′ end of region F′ (ii) or both (iii), respectively to generate designs of the following formulas:

D′-F-G-F′ (i)

F-G-F′-D″ (ii)

D′-F-G-F′-D″ (iii)

In this instance, the F-G-F′ is the gapmer portion of the oligonucleotide and region D′ or D″ constitute a separate part of the oligonucleotide. Region D′ or D″ may independently comprise or consist of 1, 2, 3, 4 or 5 additional nucleotides, which may be complementary or non-complementary to the target nucleic acid. The nucleotide adjacent to the F or F′ region is not a sugar-modified nucleotide, such as a DNA or RNA or base modified versions of these.

As described above, the D′ or D′ region may serve as a nuclease susceptible biocleavable linker.

In some embodiments, the additional 5′ and/or 3′ end nucleotides are linked with phosphodiester linkages. Nucleotide based biocleavable linkers suitable for use as region D′ or D″ are notably disclosed in WO2014/076195, which include by way of example a phosphodiester linked DNA dinucleotide. The use of biocleavable linkers in poly-oligonucleotide constructs is disclosed in WO2015/113922, where they are used to link multiple antisense constructs (e.g. gapmer regions) within a single oligonucleotide.

In a very particular aspect, the gapmer comprises, essentially consists in or consists in the sequence of Gapmer#1 (SEQ ID NO: 29) or Gapmer#2 (SEQ ID NO: 30) or a gapmer comprising at least 10 consecutive nucleotides of one of these sequences. Optionally, this gapmer is a LNA gapmer.

The present invention relates to any of the ASO described above and their use as a drug.

Pharmaceutical Composition and its Uses

The present invention relates to a pharmaceutical composition comprising any of the ASO described above and its uses as a drug, especially for the treatment of melanoma.

The pharmaceutical compositions contemplated herein may include a pharmaceutically acceptable carrier in addition to the active ingredient(s). The term “pharmaceutically acceptable carrier” is meant to encompass any carrier (e.g., support, substance, solvent, etc.) which does not interfere with effectiveness of the biological activity of the active ingredient(s) and that is not toxic to the host to which it is administered. For example, for parental administration, the active compounds(s) may be formulated in a unit dosage form for injection in vehicles such as saline, dextrose solution, serum albumin and Ringer's solution.

The pharmaceutical composition can be formulated as solutions in pharmaceutically compatible solvents or as emulsions, suspensions or dispersions in suitable pharmaceutical solvents or vehicle, or as pills, tablets or capsules that contain solid vehicles in a way known in the art. Formulations of the present invention suitable for oral administration may be in the form of discrete units as capsules, sachets, tablets or lozenges, each containing a predetermined amount of the active ingredient; in the form of a powder or granules; in the form of a solution or a suspension in an aqueous liquid or non-aqueous liquid; or in the form of an oil-in-water emulsion or a water-in-oil emulsion. Formulations suitable for parental administration conveniently comprise a sterile oily or aqueous preparation of the active ingredient which is preferably isotonic with the blood of the recipient. Every such formulation can also contain other pharmaceutically compatible and nontoxic auxiliary agents, such as, e.g. stabilizers, antioxidants, binders, dyes, emulsifiers or flavoring substances. The formulations of the present invention comprise an active ingredient in association with a pharmaceutically acceptable carrier therefore and optionally other therapeutic ingredients. The carrier must be “acceptable” in the sense of being compatible with the other ingredients of the formulations and not deleterious to the recipient thereof. The pharmaceutical compositions are advantageously applied by injection or intravenous infusion of suitable sterile solutions or as oral dosage by the digestive tract. Methods for the safe and effective administration of most of these chemotherapeutic agents are known to those skilled in the art. In addition, their administration is described in the standard literature.

The administration route for the ASO and the pharmaceutical composition comprising it as disclosed herein may be oral, parental, intravenous, intratumoral, subcutaneous, intracranial, intra-arterial, topical, rectal, transdermal, intradermal, nasal, intramuscular, intraperitoneal, intraosseous, and the like.

The pharmaceutical composition may further comprise an additional therapeutic agent. The additional therapeutic agent can be any therapeutic agent for the treatment of cancer, especially of melanoma.

More specifically, the additional therapeutic agent can be selected from the group consisting of a chemotherapeutic agent, a targeted therapy, an immune check point inhibitor, an immunotherapy, or a combination thereof. A non-exhaustive list of these additional therapeutic agents is provided below.

Accordingly, the present invention further relates to
a pharmaceutical composition comprising an ASO targeting LINC00518 as described below, an additional therapeutic agent and a pharmaceutically acceptable carrier, in particular for use in the treatment of melanoma;
a product or kit containing (a) an ASO targeting LINC00518 as disclosed below, and optionally b) an additional therapeutic agent, as a combined preparation for simultaneous, separate or sequential use, in particular in the treatment of melanoma;
a combined preparation which comprises (a) an ASO targeting LINC00518 as disclosed below, b) an additional therapeutic agent as described below for simultaneous, separate or sequential use, in particular in the treatment of melanoma;
a pharmaceutical composition comprising a conjugated nucleic acid molecule as disclosed below, for the use in the treatment of melanoma in combination with an additional therapeutic agent and/or with radiotherapy;
the use of a pharmaceutical composition comprising an ASO targeting LINC00518 as disclosed below for the manufacture of a medicament for the treatment of melanoma in combination with an additional therapeutic agent and/or with radiotherapy;
a method for treating a melanoma in a patient in need thereof, comprising administering an effective amount of a) an ASO targeting LINC00518 as disclosed below, and b) an effective amount of an additional therapeutic agent; and
a method for treating a melanoma in a patient in need thereof, comprising administering an effective amount of a pharmaceutical composition comprising an ASO targeting LINC00518 as disclosed herein, and an effective amount of an additional therapeutic agent.

Melanoma

As used herein, the term “melanoma” also known as malignant melanoma, refers to a type of cancer that develops from the pigment-producing cells, called melanocytes. There are three general categories of melanoma: 1) cutaneous melanoma which corresponds to melanoma of the skin; it is the most common type of melanoma; 2) mucosal melanoma which can occur in any mucous membrane of the body, including the nasal passages, the throat, the vagina, the anus, or in the mouth; and 3) ocular melanoma also known as uveal melanoma or choroidal melanoma, is a rare form of melanoma that occurs in the eye. In a particular embodiment, the melanoma is cutaneous melanoma.

The melanoma to be treated can be at any stage. For instance, the melanoma can be of stage I/II, II, III, or IV, preferably stage II, III, or IV, more preferably stage III, or IV. More specifically, the melanoma can be of stage T1a, T1b, T2a, T2b, T3a, T4a, T4b, N1, N2, N3, M1a, M1b, or M1c, preferably stage T2b, T3a, T4a, T4b, N1, N2, N3, M1a, M1b, or M1c. In a particular aspect, the melanoma is at an advanced or metastatic stage.

In some aspects, the melanoma is selected from the group consisting of lentigo maligna, lentigo maligna melanoma, superficial spreading melanoma, acral lentiginous melanoma, mucosal melanoma, nodular melanoma, polypoid melanoma, desmoplastic melanoma, melanoma with small nevus-like cells, melanoma with features of a Spitz nevus, uveal melanoma, Harding-Passey melanoma, juvenile melanoma, amelanotic melanoma, Cloudman's melanoma, and vaginal melanoma.

The subject may have resistant melanoma. As used herein, the term “resistant melanoma” refers to melanoma, which does not respond to a classical treatment. The cancer may be resistant at the beginning of treatment or it may become resistant during treatment or does not respond anymore. The resistance to drug leads to rapid progression of metastatic of melanoma. The melanoma can be resistant to targeted therapy, especially BRAF inhibitors, MEK inhibitors or C-Kit inhibitors, to chemotherapy, or to checkpoint inhibitors. In a preferred aspect, the melanoma is resistant to a BRAF inhibitor such as dabrafenib, vemurafenib, or trametinib. Indeed, the ASO targeting LINC00518 may restore or increase the sensitivity of melanoma to this drug. In another preferred aspect, the melanoma is resistant to chemotherapeutic agent such as temozolomide and dacarbazine. Indeed, the ASO targeting LINC00518 may restore or increase the sensitivity of melanoma to this drug.

In a particular aspect, the present invention relates to a method for increasing the sensitivity of a melanoma resistant to a therapeutic agent comprising administering a therapeutically active amount of the ASO targeting LINC00518 as disclosed herein. The method may further comprise the administration of the therapeutic agent. The therapeutic agent can be a targeted therapy, especially BRAF inhibitors, MEK inhibitors, C-Kit inhibitors, a chemotherapy, or a checkpoint inhibitor. In a preferred embodiment, the therapeutic agent is a BRAF inhibitor such as dabrafenib, vemurafenib, or trametinib. In another preferred embodiment, the therapeutic agent is a chemotherapeutic agent such as temozolomide and dacarbazine.

In an alternative aspect, the melanoma is sensitive to a targeted therapy (i.e., respond to the targeted therapy). Indeed, it is believed that the ASO targeting LINC00518 as disclosed herein is able to prevent, decrease or delay the appearance of a resistance of the melanoma to a targeted therapy. Preferably, the targeted therapy is an inhibitor of the MAPK pathway, in particular selected from the group consisting of a BRAF inhibitor, a C-Kit inhibitor, and a MEK inhibitor a, preferably a BRAF inhibitor.

The subject may have BRAF mutation. A number of mutations in BRAF are known. In particular, the V600E mutation is prominent. Other mutations which have been found are R4611, 1462S, G463E, G463V, G465A, G465E, G465V, G468A, G468E, N5805, E585K, D593V, F594L, G595R, L596V, T5981, V599D, V599E, V599K, V599R, K600E, A727V, and most of these mutations are clustered to two regions: the glycine-rich P loop of the N lobe and the activation segment and flanking regions. In a particular embodiment, the BRAF mutation is V600E/K in the context of the invention.

Alternatively, the subject may have a melanoma without BRAF mutation.

Combined Treatments

The ASO targeting LINC00518 can be used in combination with an additional therapeutic agent. In a preferred aspect, the additional therapeutic agent is an agent suitable for the treatment of cancer, especially melanoma. These agents are well-known by the person skilled in the art. For illustration, please see the review Kozar et al (BBA—Reviews on Cancer 1871 (2019) 313-322). The additional therapeutic agent can be a targeted therapy, a chemotherapy, an immune checkpoint inhibitor, an immunotherapy or a combination thereof. The additional therapeutic agent can be a siRNA, shRNA or antisense oligonucleotide targeting a mRNA, miRNA or IncRNA of interest such as those disclosed in WO19198115.

In a first aspect, the additional therapeutic agent is a targeted therapy. For instance, the therapeutic agent of targeted therapy can be a BRAF inhibitor, a MEK inhibitor, a C-Kit inhibitor, or a combination thereof. In a particular aspect, the targeted therapy can be a combination of a BRAF inhibitor and a MEK inhibitor.

The present invention relates to a pharmaceutical composition comprising an ASO targeting LINC00518 as defined herein and an inhibitor of the MAPK pathway, especially for use for treating melanoma. Optionally, the inhibitor of the MAPK pathway can be a BRAF inhibitor, a MEK inhibitor, a C-Kit inhibitor, or a combination thereof. In a particular aspect, the inhibitor of the MAPK pathway can be a combination of a BRAF inhibitor and a MEK inhibitor. It further relates to a product or kit containing (a) an ASO targeting

LINC00518 as disclosed herein, and b) an inhibitor of the MAPK pathway, as a combined preparation for simultaneous, separate or sequential use, in particular in the treatment of melanoma. It also relates to an ASO targeting LINC00518 as disclosed herein for use for treating a melanoma in combination with an inhibitor of the MAPK pathway. Optionally, the ASO targeting LINC00518 as disclosed herein and the inhibitor of the MAPK pathway are used so as to obtain a synergistic effect on apoptosis of melanoma cells. Optionally, the inhibitor of the MAPK pathway is used in a sub-therapeutic amount.

The inhibitors of BRAF are well known in the art. BRAF inhibitors are described in e.g. WO 2005/062795, WO 2007/002325, WO 2007/002433, WO 2008/079903, and WO 2008/079906, the disclosure thereof being incorporated by reference. In a particular embodiment, the inhibitor of BRAF can be Vemurafenib or other inhibitors as disclosed in U.S. Pat. Nos. 8,470,818, 8,470,818, 8,143,271, 7,863,288, 9,447,089, U.S. Pat. No. 7,504,509; 8,741,920, the disclosure thereof being incorporated by reference. Vemurafenib also known as PLX4032, RG7204 or R05185426 and commercialized by Roche as zelboraf. Alternatively, the inhibitor of BRAF can be Dabrafenib also known as tafinlar, which is commercialized by Novartis. Other inhibitors of BRAF can be encorafenib, or also LGX818 (Novartis), TAK-632 (Takeda), MLN2480 (Takeda/Millennium), PLX-4720 (Plexxikon). In particular, the examples provide evidence that the combination of the ASO targeting LINC00518 with a BRAF inhibitor is advantageous, in particular to increase the apoptosis of melanoma cells. In a particular aspect, it could be interesting for the treatment of melanoma resistant to a treatment with a BRAF inhibitor, especially dabrafenib, vemurafenib and encorafenib, more particularly vemurafenib.

The inhibitors of MEK are well known in the art. In a particular embodiment, the inhibitor of MEK can be Trametinib also known as mekinist, which is commercialized by GSK. In another particular embodiment, the inhibitor of MEK can be Cobimetinib also known as cotellic commercialized by Genentech. In a further particular embodiment, the inhibitor of MEK can be Binimetinib also known as MEK162, ARRY-162 is developed by Array Biopharma. Other MEK inhibitors can be AZD6244 (AstraZeneca/Array BioPharma), R05126766 (Roche/Chugai), GDC-0623 (Genentech/Chugai), PD0325901 (Pfizer), and Selumetinib.

The chemotherapeutic agent can be selected from the group consisting of an inhibitor of topoisomerases I or II, a DNA crosslinker, a DNA alkylating agent, an anti-metabolic agent and inhibitors of the mitotic spindles. In a particular aspect, the chemotherapeutic agent can be for instance temozolomide or any alternative alkykating drugs and dacarbazine.

The immune checkpoint inhibitors can be an antibody targeting immune checkpoint such as CTLA4, PD-1, or PD-L1 and a TKR agonist or a CD40 agonist and combination thereof. In a preferred aspect, the immune checkpoint inhibitors can be an anti-CTLA4 antibody such as ipilimumab and tremelimumab, an anti-PD-1 antibody such as pembrolizumab, pidilizumab, and nivolumab, or a combination thereof.

Other anti-PD-1 could be selected from the group consisting of Cemiplimab (Libtayo), Camrelizumab, AUNP12, AMP-224, AGEN-2034, BGB-A317 (Tisleizumab), PDR001 (spartalizumab), MK-3477, SCH-900475, PF-06801591, JNJ-63723283, genolimzumab (CBT-501), LZM-009, BCD-100, SHR-1201, BAT-1306, AK-103 (HX-008), MEDI-0680 (also known as AMP-514) MED10608, JS001 (see Si-Yang Liu et al., J. Hematol. Onco1.10:136 (2017)), B1-754091, CBT-501, INCSHR1210 (also known as SHR-1210), TSR-042 (also known as ANB011), GLS-010 (also known as WBP3055), AM-0001 (Armo), STI-1110 (see WO 2014/194302), AGEN2034 (see WO 2017/040790), MGA012 (see WO 2017/19846), or 1B1308 (see WO 2017/024465, WO 2017/025016, WO 2017/132825, and WO 2017/133540), monoclonal antibodies 5C4, 17D8, 2D3, 4H1, 4A11, 7D3, and 5F4, described in WO 2006/121168. Bifunctional or bispecific molecules targeting PD-1 are also known such as RG7769 (Roche), XmAb20717 (Xencor), MED15752 (AstraZeneca), FS118 (F-star), SL-279252 (Takeda) and XmAb23104 (Xencor).

Anti-PD-L1 inhibitor can be selected from FAZ053 (Novartis), Atezolizumab (Genentech/Roche), Avelumab (Merck Serono and Pfizer), Durvalumab (Medlmmune/AstraZeneca), or BMS-936559 (Bristol-Myers Squibb). Further known anti-PD-L1 antibodies include those described, e.g., in WO 2015/181342, WO 2014/100079, WO 2016/000619, WO 2014/022758, WO 2014/055897, WO 2015/061668, WO 2013/079174, WO 2012/145493, WO 2015/112805, WO 2015/109124, WO 2015/195163, U.S. Pat. Nos. 8,168,179; 8,552,154, 8,460,927, and 9,175,082, the disclosure thereof being incorporated by reference.

The immunotherapy can be an interferon, especially interferon α-2b (IFNα-2b), or an interleukin, especially IL-2.

Finally, the additional therapeutic agent can be an oncolytic viral therapy such as T-VEC.

Further aspects and advantages of the present invention will be disclosed in the following experimental section, which should be regarded as illustrative and not limiting the scope of the present application.

### EXAMPLES

Results

### Example 1: LINC00518 Expression in Normal Tissues and Human Tumors

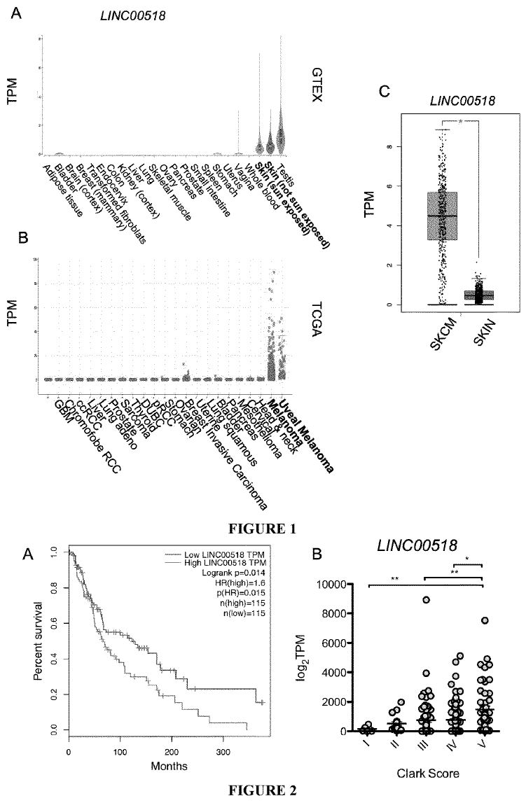

Mining of public data to assess LINC00518 expression in normal human tissues from the GTEX data base revealed that its expression is detected in the skin, the testis and at a lower level in the vagina (FIG. 1A). Expression in skin is related to the presence of melanocytes and as the inventors show below not keratinocytes. Lower expression in vagina can also be explained by the presence of low number of melanocytes in mucosal tissues. Several mucosal tissues are sites for melanoma and vaginal melanoma accounts for 0.3% of melanoma. The promiscuous and high level of expression of LINC00518 in germ cells like a large majority of genes in testis is thought to be associated with transcription coupled DNA repair as a mechanism to minimize de novo germ line mutations rather than a specific biological function of the expressed gene. The lack of LINC00518 expression in normal tissues aside melanocytes is compatible with the idea that using an ASO to target its expression in melanoma patients will minimally or not at all affect the function of normal tissues.

Mining of the public Cancer Genome Atlas database to assess LINC00518 expression in the indicated human tumours, showed that it is strongly and specifically expressed in epidermal and uveal melanoma. Aside from a weak expression in breast cancer, no significant expression is seen in other human tumours (FIG. 1B). This data shows that LINC00518 expression is highly selective in normal tissues, being expressed only in melanocytes and is strongly and selectively expressed in melanoma. After normalization of expression values between the two public data sets, a strong increase in Lin00518 expression is seen in melanoma compared to normal human skin (FIG. 1C). This results from enrichment in the melanocyte population in the melanoma samples and as the inventors show below, increased expression in transformed versus normal melanocytes.

### Example 2: LINC00518 Expression in Melanoma Correlates with Patient Outcome

Mining of the public Cancer Genome Atlas database indicated a strong positive correlation of LINC00518 expression levels with patient survival (FIG. 2A). LINC00518 expression is also positively correlated with

Clark score, a measure of advancement of primary melanoma (FIG. 2B). These data point to the clinical relevance of LINC00518 expression in human melanoma.

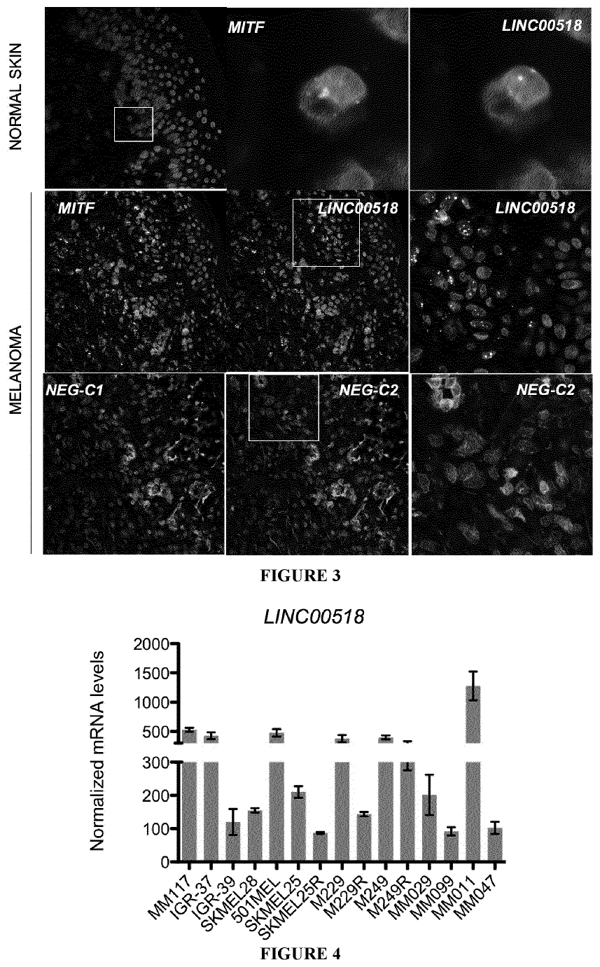

### Example 3: LINC00518 Expression is Restricted to Melanocytes (FIG. 3)

RNAscope hybridization on sections from normal human skin with a probe to detect the melanocyte-specific transcription factor MITF identified a normal melanocyte amongst the keratinocyte population. A higher magnification of this region showed low signal for MITF and LINC00518 only in the melanocyte. No expression for either gene is seen in the surrounding keratinocytes. This result confirms the melanocyte-specific expression of LINC00518. The middle panel shows RNAscope hybridization to detect MITF and LINC00518 expression in a section through a primary cutaneous melanoma. Abundant co-expression of both genes is observed in the melanoma cells. The lower panel shows a negative control demonstrating the specificity of the detected signals. These data show that LINC00518 expression is restricted to melanocytes and not keratinocytes and is strongly up regulated in melanoma.

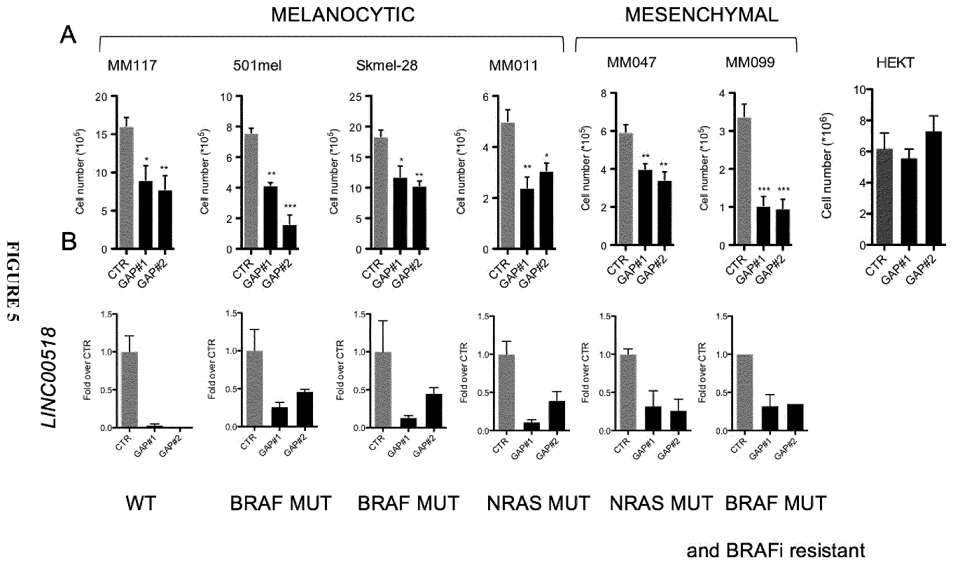

### Example 4: LINC00518 is Expressed in Melanocytic and Mesenchymal Melanoma Cell Lines (FIG. 4)

The inventors used RT-qPCR to evaluate LINC00518 expression in a collection of melanoma cells lines or primary cultures (designated MM) from patients. LINC00518 is expressed at variable levels in all tested lines with in general higher expression in differentiated melanocytic-type cells compared to undifferentiated mesenchymal-type cells. LINC00518 is therefore expressed in melanoma in vivo and in all melanoma cell lines irrespective of their mutation status or their cell phenotype.

### Example 5: LINC00518 is Essential for Melanoma Cell Line Proliferation (FIG. 5)

Melanoma cells were transfected with two independent locked nucleic acid (LNA) GapmeRs targeting LINC00518, or a control non-targeting GapmeR. 72 hours after transfection the number of viable cells was evaluated and the expression of LINC00518 evaluated by RT-qPCR. In all tested lines, irrespective of their mutations status or cell phenotype (melanocytic-type or mesenchymal-type), LINC00518 silencing led to a significantly reduced cell number. In addition, human embryonic kidney cells (HEKT) that do not express LINC00518 were used as control to evaluate the intrinsic toxicity of the GapmeRs. In agreement with the lack of expression, the GapmeRs targeting LINC00518 had no significant effect on cell proliferation in this line. The inventors thus demonstrate that LINC00518 knockdown by an ASO induces apoptosis and reduces proliferation in the undifferentiated/mesenchymal-type that are induced by and mediate resistance to targeted/immune therapy.

### Example 6: LINC00518 is Essential for Normal Melanoma Cell Cycle

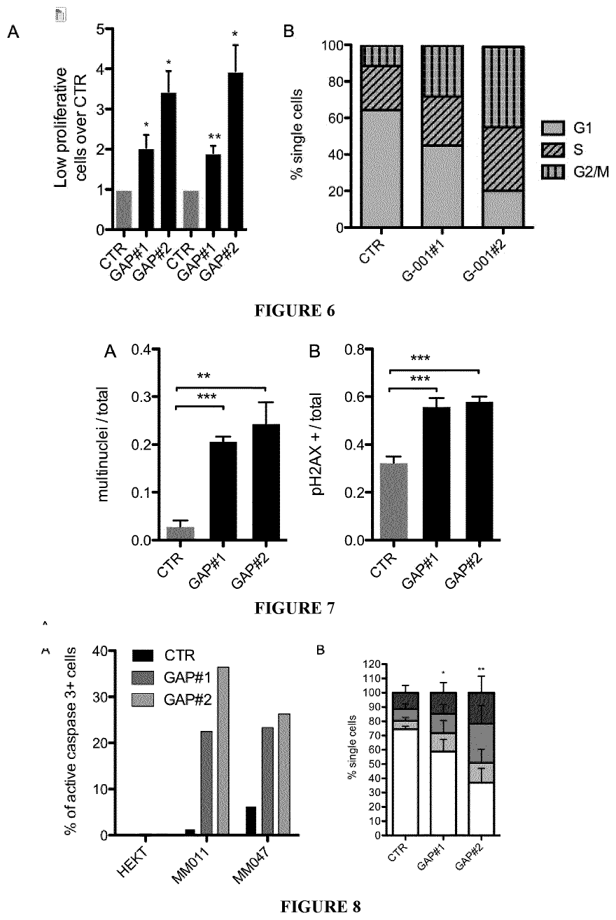

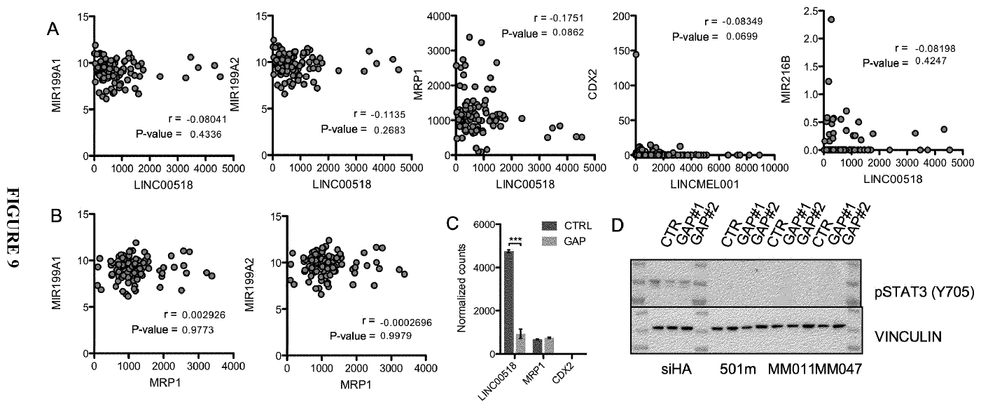

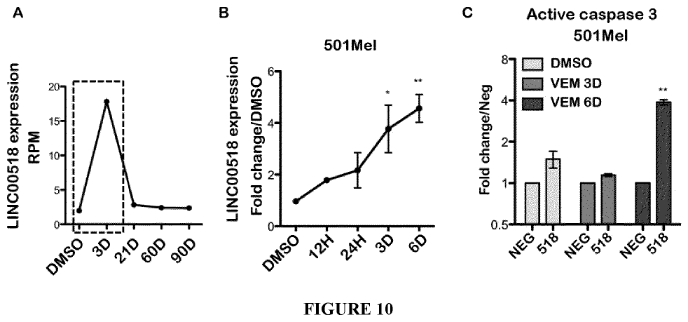

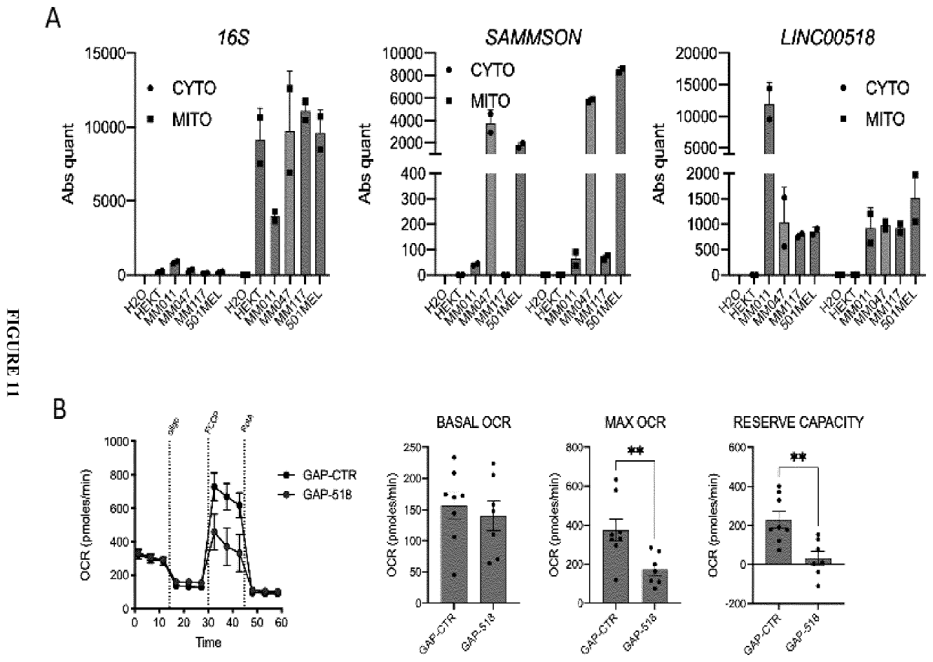

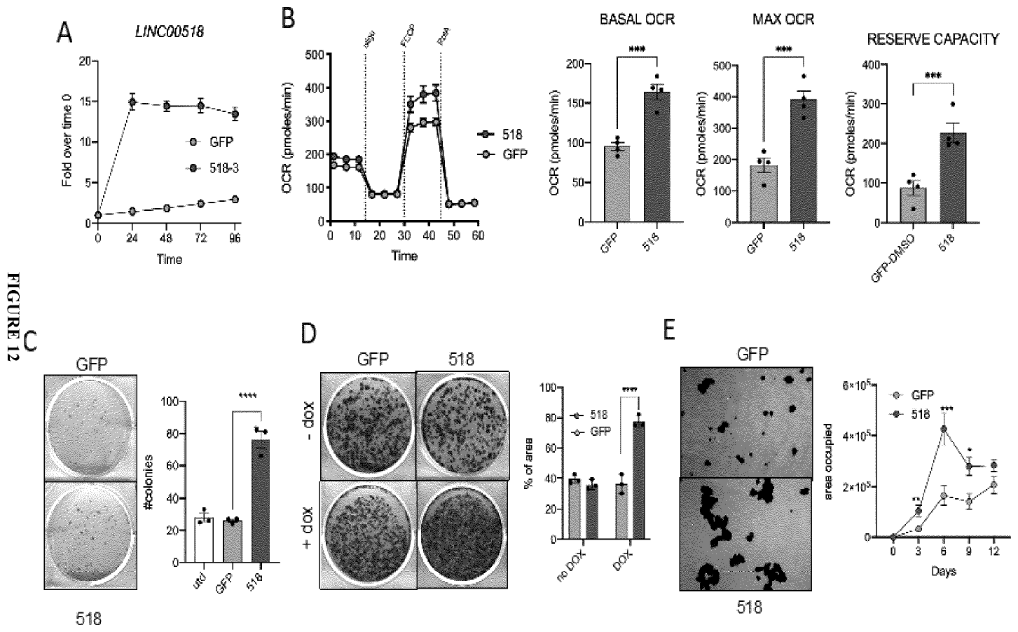

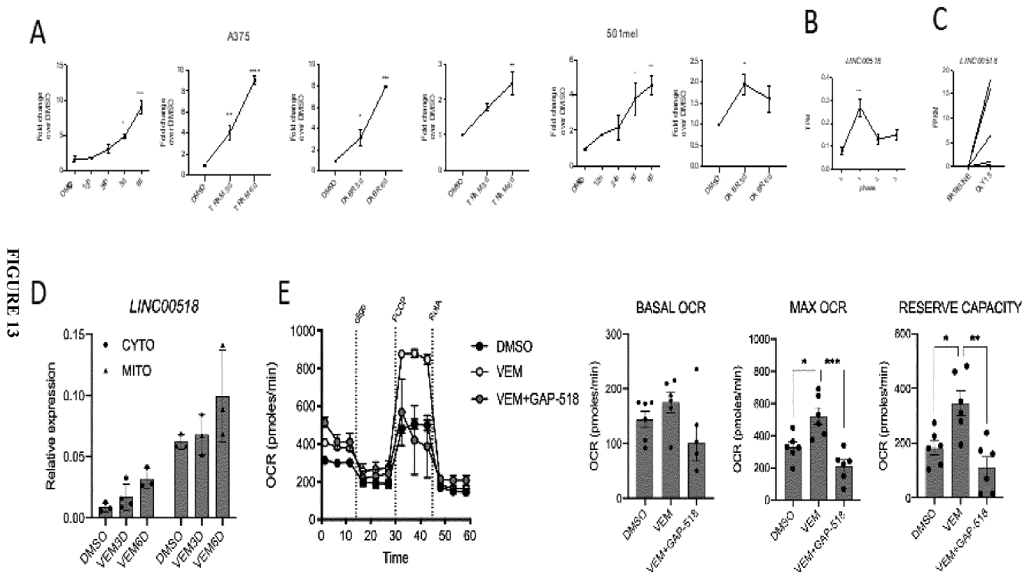

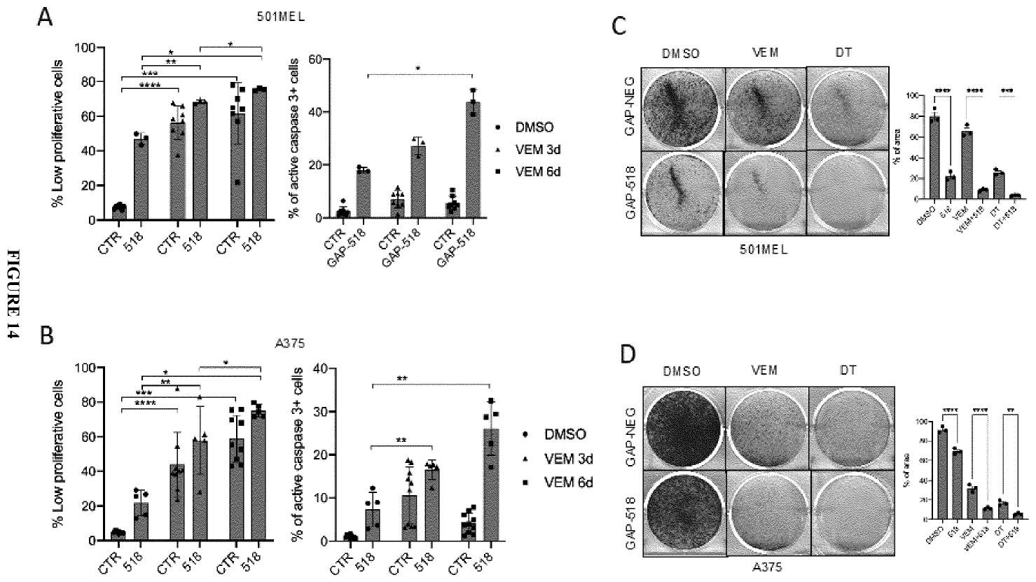

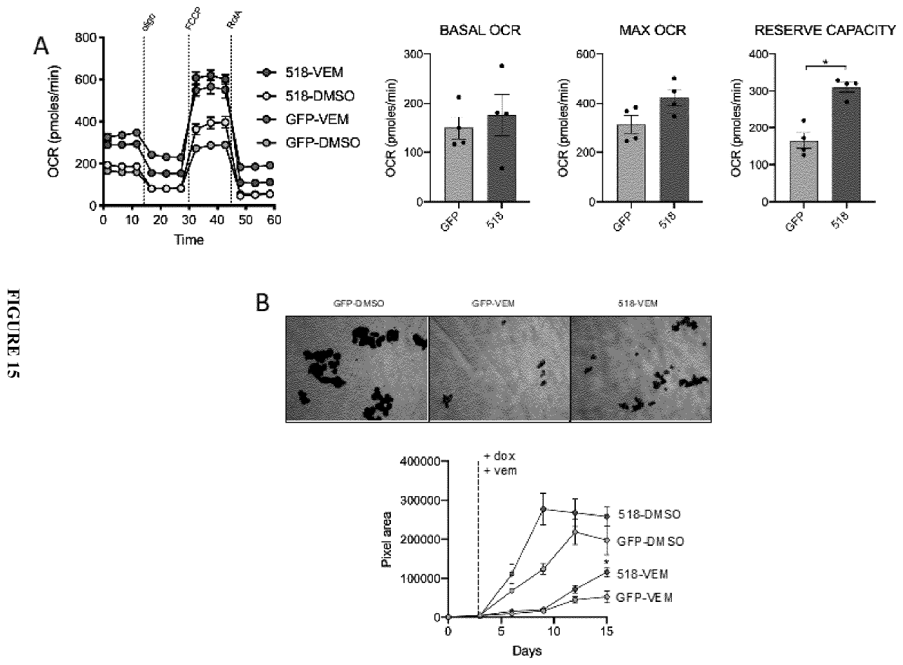

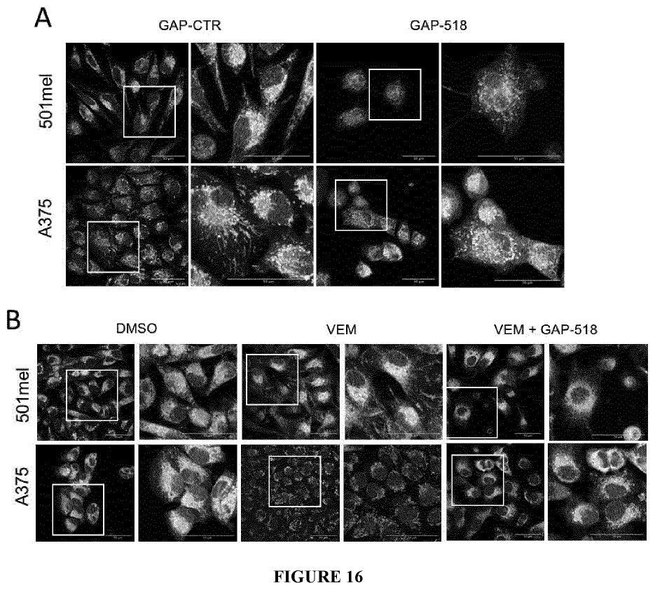

Melanoma cells were transfected with GapmeRs targeting LINC00518, or non-targeting GapmeR and cell proliferation directly assessed by Cell Trace Violet labeling and flow cytometry (FIG. 6A). LINC00518 silencing strongly increased the number of slow proliferating cells. The proportion of cells in each stage of the cell cycle was also compared showing that LINC00518 silencing led to an increased number of cells in G2/M (FIG. 6B).

### Example 7: LINC00518 is Essential for Normal Melanoma Cell Mitosis

Following transfection, the number of bi—on multi-nucleate melanoma cells was assessed showing a strong increase upon LINC00518 silencing (FIG. 7A). This increase was accompanied by an increased number of cells labelled for phospho-gamma H2AX as a marker for DNA damage (FIG. 7B).

### Example 8: LINC00518 is Essential for Melanoma Survival

Melanoma cells were transfected with GapmeRs targeting LINC00518, or non-targeting GapmeR and apoptosis assessed by flow cytometry for activation of Caspase 3 or Annexin V staining. LINC00518 led to a more than 25-fold increase in Caspase 3 labelled cells (FIG. 8A) and induced various stages of cell death in up to 70% of the cell population (FIG. 8B). GapmeR-mediated silencing therefore demonstrated that LINC00518 is essential for normal melanoma cell proliferation and survival. Its silencing leads to a G2/M arrest, induction of DNA damage, aberrant mitosis and apoptosis.

### Example 9: No Evidence for LINC00518 Acting Via miR-RNAs or Via the JAK-STAT Pathway in Melanoma

The inventors analyzed the expression of miRNAs or genes suggested to be targets of LINC00518 in cultured cell lines of other tumor types, in the patient melanoma collection of the Cancer Genome Atlas database. The inventors found no correlation between LINC00518 expression and that of mir199A/B or MRP1 in human patient melanoma samples (FIG. 9A). Similarly, CDX2 and miR16B are not expressed in melanoma. Moreover, no correlation between miR199A/B and its putative target MRP1 was seen in melanoma patient samples (FIG. 9B). Further, MRP1 expression is not affected by LINC00518 silencing in melanoma cells that show no CDX2 expression (FIG. 9C). The inventors also investigated the effect of LINC00518 silencing on JAK-STAT signaling via detection of phospho STAT3 Y705. While a mild decrease was seen in SiHa cervical carcinoma cells, no phospho STAT3 Y705 signal was seen in 3 different melanoma cell lines showing that this pathway is not active (FIG. 9D). Thus, the inventors find no evidence that the mechanism of action of LINC00518 in melanoma is related to that reported in cell lines from breast cancer or other cancers including cervical and prostate where no LINC00518 expression is detected in patient samples.

### Example 10: Vemurafenib Potentiates Melanoma Cell Apoptosis Induced by LINC00518 Knockdown

The inventors analyzed LINC00518 expression in public datasets of melanoma cells treated with the BRAF inhibitor Vemurafenib and noted that its expression was strongly up regulated by 3 days during the acute phase of the response before being down regulated. The inventors confirmed these results in Vemurafenib-treated 501Mel cells by RT-qPCR showing that LINC00518 expression was induced from 12 hours to 6 days after treatment. Cells treated for 3-6 days with 1 μm Vemurafenib or with DMSO control were transfected with suboptimal concentration (30 μM compared to 50 μM used in the other experiments) of negative control or LINC00518 GapmeR and activated Caspase 3 was measured by flow cytometry. Compared to DMSO treated cells, a strong increase in activated Caspase 3 was seen in cells with LINC00518 GapmeR after 6 days of Vemurafenib treatment. The inventors thus demonstrate that LINC00518 is induced by Vemurafenib and that Vemurafenib treatment potentiates melanoma cell apoptosis upon LINC00518 knock-down.

### Example 11: LINC00518 Localises to Mitochondria and Regulates Oxidative Phosphorylation in Melanoma Cells

The inventors performed RNA extraction from cytoplasmic and mitochondrial fractions of several melanoma cell lines and HEK293T cells and confirmed efficient separation of the two cellular compartments using RT-qPCR against the 16S mitochondrial ribosomal RNA present almost exclusively in the mitochondrial fraction (FIG. 11A). SAMMSON and LINC00518 on the other hand were present in both the cytoplasmic and mitochondrial fractions (FIG. 11A). These data showed that LINC00518 is associated with the mitochondria similar to SAMMSON previously described as a mitochondrial localized LincRNA.

The inventors next investigated the effect of LINC00518 silencing on mitochondrial activity by profiling oxidative phosphorylation (OXPHOS) in real time using the Agilent Seahorse instrument. Compared to control, LINC00518 GapmeR silencing did not affect basal oxygen consumption rate (OCR), but led to potent decrease in maximal and reserve capacity in 501Mel cells (FIG. 11B).

### Example 12: LINC00518 Over-Expression Increases Oxidative Phosphorylation and Proliferation of Melanoma Cells

As LINC00518 silencing with GapmeR impaired cell proliferation, reduced OXPHOS capacity and induced apoptosis, we assessed the effects of LINC00518 gain of function. To do this, the inventors generated a Lentiviral vector directing Doxycycline (Dox)-inducible expression of LINC00518 isoform 3, with an inducible GFP-expressing vector as control. After infection, selection and Dox treatment, RT-qPCR detected a time-dependent ectopic expression of the LINC00518 isoform (FIG. 12A). Profiling OCR in 501Mel cells with Dox-inducible expression of LINC00518 isoform 3 showed that its overexpression increased basal, maximal and reserve OCR (FIG. 12B). Ectopic LINC00518 expression increased melanoma cell colony forming capacity (FIG. 12C), 2D growth and growth as 3D melanospheres (FIGS. 12D and E). Thus, while LINC00518 silencing compromised cellular OXPHOS capacity leading to decreased melanoma cell proliferation and survival, its overexpression promoted OXPHOS and growth under 2D and 3D conditions.

### Example 13: LINC00518 is Increased Upon MAPK Inhibition In Vitro and In Vivo and Sustains Metabolic Adaptation

Mining public data sets of M229 melanoma cells treated with the BRAF inhibitor Vemurafenib (Vem) showed that LINC00518 expression was up-regulated 3 days after Vem exposure and then returned to basal level at later times. To confirm these data, the inventors measured LINC00518 expression in Vem-treated 501Mel and A375 cells by RT-qPCR confirming its up-regulation between 12-72 hours after treatment with the BRAF inhibitors Vem and Dabrafenib and the MEK inhibitor Trametinib (FIG. 13A). Similar observations were made in public data from a melanoma PDX treated in combination with Dabrafenib and Trametinib where LINC00518 expression was increased in phase 1 following inhibitor treatment (FIG. 13B). Moreover, in triple wild-type patients where neither BRAF nor NRAS were mutated, treatment with a Durvalumab-Trametinib immune checkpoint-MEK inhibitor combination, the inventors also observed up-regulated LINC00518 expression (FIG. 13C). Together, this data indicated that LINC00518 expression was rapidly induced upon inhibition of MAPK signalling either by inhibition of BRAF or MEK or both. Moreover, upon Vem treatment, LINC00518 accumulated in the mitochondrial fraction (FIG. 13D).

Inhibition of MAPK signalling in melanoma cells inhibits glycolysis inducing a metabolic switch to OXPHOS. Given the ability of LINC00518 to stimulate OXPHOS, its rapidly increased expression upon Vem treatment suggested that LINC00518 may play an important role in stimulating OXPHOS to maintain cell survival at this early stage, and hence that Vem-treated cells may display enhanced sensitive to LINC00518 silencing. To investigate this idea, the inventors profiled OXPHOS in DMSO/Vem-treated cells with or without

LINC00518 silencing. Vem increased basal, reserve and maximal OCR compared to DMSO control (FIG. 13E). Increased OCR was strongly diminished in cells silenced for LINC00518 showing its essential role in the adaptive metabolic switch (FIG. 13E).

### Example 14: LINC00518 Inhibition Synergizes with MAPK Pathway Targeting to Induce Apoptosis

Vem treatment induced cell cycle arrest with a strong increase in the number of slow proliferating control cells after 3 and 6 days, that was not further increased by LINC00518 silencing (FIG. 14A). On the other hand, LINC00518 silencing induced slowed proliferation of control DMSO treated cells (FIG. 14A). In contrast, Vem did not appreciably induce apoptosis during this period, but Vem-treated cells displayed increased apoptosis compared to DMSO-treated cells after LINC00518 silencing (FIG. 14B). The enhanced sensitivity of the Vem treated cells was clearly seen in cells treated for 6 days with Vem or DMSO where there was a synergistic increase in apoptosis upon LINC00518 knockdown. Similar to Vem, Dabrafenib and Trametinib treated cells were also more sensitive to LINC00518 silencing (FIGS. 14C and D). Thus, Vem and other inhibitors of BRAF and MAPK signalling cooperated with L518 silencing to induce melanoma cell apoptosis.

### Example 15: LINC00518 Over-Expression Reduces Vemurafenib Anti-Proliferative Effect

In further support of the idea that MAPK inhibition sensitizes melanoma cells to apoptosis after LINC00518 knockdown, the inventors assessed OXPHOS in cells with Dox-inducible ectopic expression of GFP or LINC00518 in the presence of Vem. Compared to GFP control cells, ectopic LINC00518 expression further increased OCR above the increase already seen with Vem alone indicating a stronger stimulation of the adaptive metabolic response (FIG. 15A). In line with this, the inhibition of melanosphere formation seen by Vem treatment of control GFP-expressing cells was rescued in cells with ectopic LINC00518 expression (FIG. 15B). Increased endogenous LINC00518 expression as seen after BRAF inhibition or ectopic LINC00518 expression therefore promoted survival of Vem-treated cells. The loss and gain of function experiments underscore the essential role of LINC00518 in the adaptive response to BRAF and MAPK inhibition.

### Example 16: LINC00518 Silencing Increased Mitochondrial Fission

Activation of MAPK signalling during oncogenic transformation stimulates mitochondrial fission associated with high anaerobic glycolysis and reduced mitochondrial activity, whereas BRAF inhibition induces an increase in fused mitochondria associated with the metabolic switch to OXPHOS. In accordance with the reduced OXPHOS, LINC00518 silencing increased mitochondrial fission leading to a switch from more elongated fused mitochondria seen in control cells to smaller and rounder mitochondria in the knockdown cells (FIG. 16A). The defective mitosis with bi- and multi-nucleate cells can be clearly seen following LINC00518 silencing. Vem-treated cells displayed predominantly elongated mitochondria, but this effect was dampened when LINC00518 was additionally silenced (FIG. 16B). Thus, LINC00518 drives the adaptive increase of OXPHOS by promoting mitochondrial fusion.

Material and Methods

Public RNA Sequencing Datasets Analyses

LINC00518 total RNA sequencing data were extracted from The Genotype-Tissue Expression (GTEX) and The Cancer Genome Atlas (TCGA) to evaluate expression across normal tissues and tumour samples. Comparison of LINC00518 expression between normal skin and cutaneous melanoma and Kaplan Meyer analysis of overall survival of melanoma patients were performed using the GEPIA web portal (Tang, Z. et al. (2017); GEPIA: a web server for cancer and normal gene expression profiling and interactive analyses. Nucleic Acids Res, 10.1093/nar/gkx247).

Cell Culture and GapmeR Transfections

Melanoma cell lines Sk-mel-25, Sk-mel-25R, Sk-mel-28 and 501me1 were grown in RPMI 1640 medium supplemented with 10% Fetal Calf Serum (FCS) and gentamicin; IGR-37 and IGR-39 in RPMI 1640 medium supplemented with 15% FCS and gentamicin. MM011, MM117, MM047, MM099 were grown in HAM-F10 medium supplemented with 10% FCS, 5.2 mM glutamax, 25 mM Hepes and penicillin/streptomycin (7.5 ug/ml). Hermes-3A cells were grown in RPMI 1640 medium supplemented with 10% FCS, 200 nM TPA, 200 μM cholera toxin, 10 ng/ml human stem cell factor (Invitrogen), 10 nM endothelin-1 (Bachem, Bubendorf, Switzerland), and penicillin/streptomycin (7.5 μg/ml). To assess cell growth and viability cells were stained with Trypan Blue (Invitrogen). M229, M229R, M249, M249R were grown in DMEM medium supplemented with glucose (4.5 g/1), 5% FCS and penicillin/streptomycin (7.5 ug/ml). A375 cells were grown in DMEM medium supplemented with glucose (4.5 g/1), 10% FCS and gentamicine. HEK293T cells were grown in DMEM medium supplemented with glucose (1 g/1), 10% FCS and penicillin/streptomycin (7.5 ug/ml). To assess cell growth and viability cells were stained with Trypan Blue (Invitrogen). Vemurafenib (PLX4032), Trametinib (GSK1120212), Dabrafenib (GSK2118436) were purchased from Selleckchem.

For GapmeR experiments cells were transfected using Lipofectamine RNAiMAX (Invitrogen) according to manufacturer's instructions with 20 or 25 nM (or other indicated concentrations) of GapmeR (Qiagen) and harvested 48 or 72 hours after. For Vemurafenib/Trametinib+Dabrafenib-GapmeR co-treatment, cells were cultured for 3 days in presence or absence (DMSO only) of Vemurafenib (1 uM), transfected with 15 nM of control GapmeR, L518 GAP#2, then cultured for additional 3 days before harvesting, RNA and protein extraction, flow cytometry analysis or fixed and stained with crystal violet.

Clonogenicity was assessed by plating 500 melanoma cells/9.6 cm2, keeping them in culture for 10 days and finally fixing in formalin and staining the colonies with 0.05% Crystal Violet solution (Sigma Aldrich).

Proliferation and Viability Analyses by Flow Cytometry

To assess cell viability and proliferation after lincRNA knock down by GapmeRs, cells were stained with Cell Trace Violet (Invitrogen) on the day of transfection. They were harvested after 96 hours and stained with active Caspase-3 (BD Biosciences) or with Annexin-V (Biolegend) and Topro-3 (Invitrogen) following manufacturer's instruction. Cells were analysed on a LSRII Fortessa (BD Biosciences) and data were analysed with Flowjo software (Tree Star).

Cell Cycle Progression Analysis by Flow Cytometry

Cells were transfected with GapmeRs and 72 hours after stained using the Click-it Edu kit (Thermofischer) and propidium iodide following manufacturer instructions. Briefly, cells were cultured with 10 uM Edu for 1 hour and 30 minutes, harvested by trypsin, washed once in 1% BSA-PBS and fixed in 4% PFA for 15 minutes at room temperature. After a wash in 1% BSA-PBS cells were permeabilized with saponin-permeabilization buffer for 15 minutes and then stained with the Click-it reaction cocktail for 30 minutes. Finally, they were washed once in 1% BSA-PBS, resuspended in 500 ul of PBS with 200 ug/ml RNAse and 10 ug/ml propidium iodide and left for 30 minutes at room temperature. Cells were analysed on a LSRII Fortessa (BD Biosciences) and data were analysed with Flowjo software (Tree Star).

Immunofluorescence Staining of Beta-Tubulin and Gamma-H2AX

Cells were grown on glass slides in 24-well plates, transfected with GapmeRs and fixed 72 hours after with 4% paraformaldehyde for 15 minutes. After two washes with PBS buffer they were permeabilized in PBS+triton X-100 0,1% for 5 minutes and blocked with PBS+10% FCS inactivated for 20 minutes. Primary antibodies were incubated overnight at 4° C. and after three washes with PBS+Triton 0,1%, cells were stained for 1 hour at room temperature with AlexaFluor-488 conjugated secondary antibodies (Life technologies) diluted 1/500 in PBS+10% FCS. After three washes with PBS+Triton 0,1%, cells were stained with DAPI (final concentration 1 ug/ml) and mounted on microscopy slides. Anti-tubulin (Abcam, ab6046) and anti-gamma H2AX (Ser139, Abcam, ab11174) antibodies were diluted 1/200 and 1/400 in PBS+10% FCS respectively.

RNA preparation and sequencing and quantitative PCR analyses

Total mRNA isolation was performed using the Genelute Mammalian Total RNA Miniprep Kit (Sigma) following manufacturer instructions, or using Trizol and isopropanol precipitation. Isolation of cytosolic, nuclear soluble and chromatin associated RNA was performed as described in (Conrad and ørom, Enhancer RNAs: Methods and protocols, 2017, 1468, 1-9). Briefly, cells were harvested and washed in PBS buffer, resuspended in 0.15% NP-40 lysis buffer and centrifuged on a 24% sucrose cushion (taking supernatant as the cytosolic fraction). Nuclei were resuspended in 1M Urea, 1% NP-40 lysis buffer and centrifuged to recover the nuclear soluble fraction in the supernatant. The chromatin pellet was finally resupended in 1m1 of Trizol reagent (MRCgene), solubilized using a 21-gauge needle and isolated following manufacturer instructions. Cyosolic and nuclear soluble fractions were cleared by centrifugation and RNA was isolated from 200 ul of each using 1 ml of Trizol. Total and fractionated RNAs were treated with DNAsel following the TurboDnase free kit instructions (Thermofisher) and reversed transcribed using Superscript IV reverse transcriptase (Thermofisher) following manufacturer instructions. qRT-PCR was carried out with SYBR Green I (Roche) and monitored by a LightCycler 480 (Roche). Target genes expression was normalized using TBP, HBMS, GAPDH, ACTB, Rpl13a as reference genes. Actin and Xist were taken as RNA controls for cellular fractionation for cytosolic and nuclear soluble/chromatin associated fraction, respectively and the mitochondrial transcribed 16S ribosomal RNA as a control for mitochondrial purity.

RNAscope

RNAs for LINC00518 and MITF in sections of human melanomas or normal skin were detected with the RNAscope assay (Advanced Cell Diagnostics, ACD, Hayward, Calif.) according to the manufacturer's protocols. Briefly, patient sections were de-paraffinized, incubated with hydrogen peroxide at room temperature for 10 min, boiled with target retrieval reagent for 15 min, and then treated with protease plus reagent at 40° C. for 30 min. The sections were hybridized with Hs-MITF probe (ACD, Cat. No. 310951) at 40° C. for 2 h. Probes for Hs-LINC00518 were custom designed by ACD. Hybridization signals were amplified and visualized with RNAscope Multiplex Fluorescent Reagent Kit v2 (ACD, Cat. No. 323100). Images were captured with a confocal (Leica DM16000) microscope.

Mitotracker Live Imaging

Cells were cultured in 4 wells 35×10 mm dishes (CellView, Greiner Bio-one), stained for 2 hours with Mitotracker Red CMXRos (125 nM) and Hoechst 33342 (1 ug/ml) and analyzed with a confocal (Leica DM16000) microscope in a temperature controlled (37° C.) chamber.

Protein Extraction and Western Blotting

Whole cell extracts were prepared by the standard freeze-thaw technique using LSDB 500 buffer (500 mM KCl, 25 mM Tris at pH 7.9, 10% glycerol (v/v), 0.05% NP-40 (v/v), 16 mM DTT, and protease inhibitor cocktail). Cell lysates were subjected to SDS—polyacrylamide gel electrophoresis (SDS-PAGE) and proteins were transferred onto a nitrocellulose membrane. Membranes were incubated with primary antibodies in 5% dry fat milk and 0.01% Tween-20 overnight at 4° C. The membrane was then incubated with HRP-conjugated secondary antibody (Jackson ImmunoResearch) for 1h at room temperature, and visualized using the ECL detection system (GE Healthcare). Antibodies used are: anti-phospho-STAT3 (Y705) from Cell Signalling Technologies (#9131) and anti-vinculin from Sigma (V9131).

Area Occupation Assay

Cells were seeded at low density (10 000 cells/9.5 cm²) and transfected with negative control or LINC00518 specific GapmeRs. One week later they were fixed with 4% paraformaldehyde and stained with crystal violet. Whole wells were scanned and the pictures analysed with Image) to calculate the % of area occupied.

Melanosphere Formation Assay

501me1 cells were plated in 10 cm petri dishes without any coating in KO DMEM medium supplemented with 25% KSR, AANE, 2.5 mM Glutamax, 125 ug/ml Penicillin/Streptomycin and 50 mM Beta-mercaptoethanol. Every three days pictures of 10 different areas uniformly distributed across the petri were taken by light microscopy. Images were analyzed by Image) to quantify the area occupied by melanospheres in each picture and the corresponding values used to calculate the mean and standard deviation for each sample.

Plasmid Cloning and Lentiviral Transduction

L518 isoform 3 cDNAs was synthesized by Genscript. L518 cDNA was cloned into the pCW57-GFP-P2A-MCS vector (a gift from Adam Karpf; Addgene plasmid #71783; http://n2t.net/addgene:71783; RRID:Addgene_71783). Lentiviral particles were produced after transfection of HEK293T cells with packaging plasmids, purified by ultracentrifugation and resuspended in PBS. After titration, melanoma cells were infected at MOI of 1 and selected by puromycin addition to the media (1 ug/ml).

Analysis of Oxygen Consumption Rate (OCR) in Living Cells

OCR was measured in an XF96 extracellular analyzer (Seahorse Bioscience). A total of 20000 transfected cells per well were seeded 48 hours prior the experiment. The cells were incubated at 37° C. and the medium was changed to XF base medium supplemented with 1 mM pyruvate, 2 mM glutamine and 10 mM glucose for 1 hour before measurement. For OCR profiling, cells were treated following the Mitostress test kit instructions and sequentially exposed to 2 μM oligomycin, 1 μM carbonyl cyanide-4- (trifluorome-thoxy) phenylhydrazone (FCCP), and 0.5 μM rotenone and antimycin A. After measurement, cells were washed with PBS, fixed with 3% PFA, permeabilized with 0.2% triton. Nuclei were counterstained with Dapi (1:500) and number of cells per well determined by the IGBMC High Throughput Cell-based Screening Facility (HTSF, Strasbourg).

Mitochondria Fractionation

Mitochondria were isolated with the Mitochondria Isolation kit (Thermofisher) following manufacturer instructions. Briefly, harvested cells were washed and pelleted, resuspended in buffer A and incubated 2 minutes on ice. Buffer B was added for 5 minutes, vortexing every minute and diluted with buffer C (same volume of buffer A). Nuclei were pelleted 10 minutes at 700 g and supernatant centrifuged for 15 minutes at 3000 g. Purified mitochondria were washed once in buffer C and used for RNA (Trizol-isopropanol precipitation) or protein (TBS+CHAPS 2%) extraction.

1-21. (canceled)

22. A method of treating melanoma comprising the administration of an antisense oligonucleotide inhibiting expression of LINC00518 to a subject having a melanoma.

23. The method according to claim 22, wherein the expression of LINC00518 is inhibited in the nucleus, the cytoplasm or the mitochondria.

24. The method according to claim 22, wherein the antisense oligonucleotide increases cancer cell apoptosis.

25. The method according to claim 22, wherein the antisense oligonucleotide decreases cancer cell proliferation.

26. The method according to claim 22, wherein the antisense oligonucleotide decreases the appearance of resistance to targeted therapy, chemotherapy or immune checkpoint therapy.

27. The method according to claim 22, wherein the melanoma is a melanoma resistant to targeted therapy, chemotherapy or immune checkpoint therapy.

28. The method according to claim 22, wherein the melanoma is an advanced melanoma or a metastatic melanoma.

29. The method according to claim 22, wherein the antisense oligonucleotide is administered in combination with a therapeutic agent used for the treatment of melanoma.

30. The method according to claim 29, wherein the therapeutic agent used for the treatment of melanoma is selected from the group consisting of a BRAF inhibitor, a C-Kit inhibitor, and a MEK inhibitor.

31. The method according to claim 29, wherein the therapeutic agent used for the treatment of melanoma is selected from the group consisting of dabrafenib, vemurafenib, encorafenib, trametinib, and binimetinib.

32. The method according to claim 29, wherein the therapeutic agent used for the treatment of melanoma is to be administered at a sub-therapeutic amount.

33. The method according to claim 29, wherein the therapeutic agent used for the treatment of melanoma is selected from the group consisting of a chemotherapy and immunotherapy.

34. The method according to claim 29, wherein the therapeutic agent used for the treatment of melanoma is selected from the group consisting of temozolomide, dacarbazine, an anti-PD-1 antibody, pembrolizumab, pidilizumab, nivolumab, an anti-CTLA-4, ipilimumab, tremelimumab, a TKR agonist, a CD40 agonist and an anti-PD-L1 antibody.

35. The method according to claim 22, wherein the antisense oligonucleotide induces a RNase H mediated degradation.

36. The method according to claim 22, wherein the antisense oligonucleotide is a Gapmer, a LNA gapmer, a MOE gapmer, a mixed wing Gapmer or an alternating flank gapmer.

37. The method according to claim 22, wherein the antisense oligonucleotide comprises a contiguous nucleotide sequence of 10 to 30 nucleotides in length wherein the contiguous nucleotide sequence is at least 90 percent complementary to exon 4 of LINC00518.

38. The method according to claim 22, wherein the antisense oligonucleotide comprises the sequence of Gapmer#1 (SEQ ID NO: 29) or Gapmer#2 (SEQ ID NO: 30) or a gapmer comprising at least 10 consecutive nucleotides of one of these sequences.

39. The method according to claim 22, wherein the antisense oligonucleotide comprises a contiguous nucleotide sequence of 10 to 30 nucleotides in length wherein the contiguous nucleotide sequence is at least 90 percent complementary to exon 1, 2 or 3 of LINC00518.

40. A method of decreasing or delaying resistance to a targeted therapy comprising the administration of an antisense oligonucleotide inhibiting expression of LINC00518 to a subject undergoing treatment with a targeted therapy.

41. The method according to claim 40, wherein the targeted therapy is selected from the group consisting of a BRAF inhibitor, a C-Kit inhibitor, and a MEK inhibitor.

42. The method according to claim 40, wherein the targeted therapy is selected from the group consisting of dabrafenib, vemurafenib, encorafenib, trametinib, and binimetinib.
